(19) United States
(12) Patent Application Publication
(10) Pub. No.: US 2005/0250691 A1
(43) Pub. Date: Nov. 10, 2005

(54) Immunomodulation by a therapeutic medication intended for treatment of diabetes and prevention of autoimmune diabetes

(75) Inventors: John Robertson, Stockholm (SE); Anders Essen-Moller, Stockholm (SE)

Correspondence Address: ROGER A. GILCREST, 250 WEST STREET, COLUMBUS, OH, 43216-7513, US

(73) Assignee: Diamyd Therapeutics AB, Stockholm (SE)

(21) Appl. No.: 10/842,715

(22) Filed: May 10, 2004

(51) Int. Cl. A61K 38/22

(52) U.S. Cl. 514/12

(57) ABSTRACT

The present invention regards methods and formulations for the treatment of diabetes and the prevention of autoimmune diabetes. The invention includes the administration of human recombinant GAD65 protein in a pharmaceutically acceptable adjuvant.

### CROSS-REFERENCE TO RELATED APPLICATIONS

This application is a continuation-in-part of U.S. application Ser. No. ______, filed Mar. 19, 2004 which claims the benefit of U.S. Provisional Application of the same title and inventorship, Ser. No. ______ filed Mar. 4, 2004, which are incorporated herein by reference.

### BACKGROUND OF THE INVENTION

Diabetes is a major cause of morbidity and mortality in industrialized societies. It has been estimated that one of every seven health-care dollars goes to treating diabetes and its complications. Type 1 diabetes (also called insulin-dependent or juvenile diabetes, henceforth referred to in this document as “diabetes”) is due to the autoimmune destruction of the insulin-producing pancreatic beta cells. Type 1 diabetes is less common than type 2, accounting for only 10-20% of cases in Caucasians. However, because it starts much earlier in life, it accounts for a large proportion of diabetes-related morbidity and mortality.

Autoimmune destruction of the pancreatic islet beta cells is the major cause of type 1 diabetes mellitus.¹ This destruction is associated with cellular and humoral immune responses to several beta-cell autoantigens, both of which can precede clinical onset of disease. Indeed, the presence of antibodies against glutamic acid decarboxylase (GADA), insulinoma-associated antigen (IA-2A) or insulin (IAA) alone or in combination has been shown to predict type 1 diabetes.^{2,3} Together with islet-cell antibodies (ICA), IA-2A and GADA are present at the time of diagnosis in 80-90% of patients with type 1 diabetes. These autoantibodies, especially GADA, may also occur in up to 10% of adults initially classified as type 2 diabetes, a condition referred to as Latent Autoimmune Diabetes in Adults (“LADA”).⁵ The disease process in LADA patients is in some ways similar to that in type 1 diabetes in that they share some HLA genetic susceptibility and some several type 1 diabetes-associated autoantibodies, although progression to insulin dependence may be slower.^{6,7}

Preclinical studies in the spontaneously non-obese type 1 diabetic (NOD) mouse demonstrated that the destruction of pancreatic islet beta-cells was associated with T cells recognising GAD65.⁸ It was also shown that small quantities of GAD65 effectively prevented autoimmune beta-cell destruction and reduced or delayed the development of spontaneous diabetes.⁸⁻¹⁴

Given the present irreversibility of the destruction of pancreatic beta cells in autoimmune diabetes, it is desireable to provide a treatment where it cannot be otherwise prevented. Although the specific causative autoantigen(s) in diabetes is/are not known, insulin and GAD 65, appear to be an autoantigen of major importance. Accordingly, it is desireable to have safe and efficacious medications and treatment and prevention methods and regimes for down-regulating beta cell destruction in autoimmune diabetes.

Based on the pre-clinical data presented herein, controlled clinical studies were initiated to assess the potential of recombinant human GAD65 to halt beta-cell destruction and prevent or reduce insulin dependence. Following extensive pre-clinical safety evaluation and one clinical phase I trial with the bulk rhGAD65 without adjuvant, a phase II study with rhGAD65 formulated with alum (the medication in accordance with the present invention) was conducted in LADA-patients. The study objectives were to investigate the clinical safety of the subcutaneously administered medication of the present invention, and to assess its impact on the immune system and diabetic status, and to identify an immunmodulatory dose level.

### SUMMARY OF THE INVENTION

The present invention relates to methods and formulations for preventing autoimmune diabetes and for treatment of human diabetes in general.

In general terms, the invention includes a method of treating diabetes in a human comprising administering to a the human an effective amount of a human recombinant GAD65 protein and at least one adjuvant for an effective time so as to stimulate the production of insulin in the human to a level above that existing prior to the administration. The method of the present invention may also be expressed as a method of improving HbA1c blood sugar values in a human, or as a method of increasing the time to insulin dependence.

The administration may be by any acceptable means, such as by subcutaneous injection or use of an implant, as well as transdermally, through nasal inhalation, intramuscular injection, colorectal administration, adeno-associated virus or DNA guns.

The adjuvant may be any pharmaceutically acceptable adjuvant substance, such as aluminum hydroxide.

The human recombinant GAD65 protein is administered in a dosage such that the human recombinant GAD65 protein is at a level of at least about 4 micrograms, preferably in the range of from about 10 micrograms to about 500 micrograms, and most preferably in the range of from about 10 to about 50 micrograms.

Following the first administration of the human recombinant GAD65 protein, additional booster dosages may be given over a treatment period (typically 2-24 weeks), preferably at a level of at least about 4 micrograms and most preferably in the range of from about 10 micrograms to about 500 micrograms.

The invention also includes a method of suppressing, regulating or reducing the immune response of a human to glutamic acid decarboxylase comprising administering to the human an effective dose of human recombinant GAD65 protein, so as to help prevent autoimmune diabetes.

The administration methods, adjuvants, dosage and booster levels and ranges may be as given above.

The invention also includes a pharmaceutical composition for regulating or reducing the immune response to a human to glutamic acid decarboxylase comprising a dosage form whose components comprise an effective dose of human recombinant GAD65 protein and a pharmaceutically acceptable adjuvant.

The method of the present invention thus also includes generally a method to increase insulin production in a diabetes patient with beta cell antibodies, the method comprising administering to a human an effective amount of beta cell antigens in a pharmaceutical carrier for an effective time so as to stimulate the production of insulin in the human to a level above that existing prior to the administration.

The beta cell antigens that may be used in the method of the present invention include at least one from the group: GAD65, GAD67, insulin, insulin-peptide, proinsulin, proinsulinpeptide, sulfatide, heat schock protein, S100 beta protein, IA-2, or any peptide, altered peptide ligand, chimeric molecule, or conjugated molecule, or fragment of any of the above.

The aforementioned methods may be practiced by replacing at least one of the beta cell antigens with DNA or RNA nucleotides coding for at least one from the group: GAD65, GAD67, insulin, insulin-peptide, proinsulin, proinsulinpeptide, sulfatide, heat schock protein, S100 beta protein, IA-2, or any peptide, altered peptide ligand, or by anti-sense oligos to at least one of the nucleotide.

These methods may be carried out with any exprexssion system whereby at least one of the aforementioned components are produced recombinantly, preferrably in a prokaryotic expression system capable of posttranslational palmitoylation. This may be done with any appropriate expression system, such as for instance baculovirus grown in Spodotera frugiperda 9 (Sf9) cells.

The administration of the antigen may be by any effective and appropriate method, such as subcutaneous administration, intravenous administration and oral administration; or by gene therapy.

Although all effective amounts are included in the subject disclosure, it is typical, and preferred, that each of the administered components are administered in a dosage such that at least one of the components is in the range of from about 5 micrograms to about 100 micrograms when given subcutaneously or, in other terms, in the range of from about 0.001 mgs/kg to about 0.1 mgs/kg when given intravenously.

The method of the present invention may also include the optional administering of at least one booster dosage of the components following the first administration, and wherein the booster is administered in a dosage such that at least one of the components is in the range of from about 5 micrograms to about 100 micrograms when administered subcutaneously, and most preferably in the range of from about 10 to about 50 micrograms.

The one booster dosage(s) of the components preferrably is/are administered in a dosage such that at least one of the components is in the range of from about 0.001 mgs/kg to about 0.1 mgs/kg when administered intraveneously.

The method of the present invention may also be described as a method to treat beta cell inflammation by means of in vivo increase of the number of regulatory CD4+CD25+ T cell subsets. This may be done as a method by means of administering an effective amount of at least one components described above.

The invention also includes a pharmaceutical composition for treatment of diabetes comprising of at least one of the aforedescribed components where at least one of the components is produced according to the methods of the present invention described herein.

The invention also features a pharmaceutical composition as described herein, preferrably wherein a Zwittergent is included in a concentration relation to at least one of the components in a relative ratio of between about 1:1 to about 1:8. The preferred pharmaceutical composition includes a pharmaceutical adjuvant such as alum, and, in another embodiment a species specific serum albumin, such as human serum albumin.

One of the findings are that an effective dosing regimen, such as a 20 microgram dose prime and boost regimen, improves beta cell function in most patients and that this can be verified by looking at an increase in a subset of CD4+ lymphocytes namely the CD4+CD25+ lymphocytes. The increase may be measured in absolute terms of CD4+CD25+ or in relative terms such as the quotient CD4+CD25+/CD4+CD25−.

If an increase of CD4+CD25+ cells is not seen a reboost may be given. If no increase another reboost. In fact to look for an increase in CD4+CD25+ cells is a way to look for efficacy of other treatments for other autoimmune diseases as well.

To minimize side effects, it may be important that the t cell receptors of the increased CD4+CD25 +population is as antigen-specific as possible. This would enable specific immunoregulation with regard to tissues expressing corresponding antigens without compromising other important immune functions such as, for example, the anti-tumor defense.

It has also been found that the medication of the present invention not only maintained the beta cells' capacity to produce insulin (measured as C-peptide) which was expected—but indeed, unexpectedly did the insulin production increase significantly (measured as c-peptide). This means that the present invention may have allowed beta cells to regenerate and survive to produce more insulin. It may also mean that the present invention may have turned off the inflammation in the beta cells and thus cleared the milieu so that increased insulin production was allowed. So the present invention may now be used as a treatment for type 1 and type 2 diabetes, not only a vaccine to prevent type 1 diabetes.

A study of the treatment in acorodnace with the present invention determined that alum-formulated human recombinant GAD65 given to patients with Latent Autoimmune Diabetes in Adults (LADA) is safe and does not compromise beta cell function, and was aimed at identifying an immunomodulatory dose for further clinical development.

This study was conducted as a randomised, double blind, placebo-controlled, dose-escalation clinical trial in a total of 47 LADA patients who received either placebo or 4, 20, 100 or 500 μg of the medication in accordance with one embodiment of the invention with subcutaneous injections at weeks one and four. Safety evaluations including neurology, beta-cell function tests, diabetes status assessment, haematology, biochemistry and cellular and humoral immunological markers were repeatedly assessed over 24 weeks.

None of the patients had significant study-related adverse events. Fasting c-peptide at 24 weeks increased compared to placebo (p=0.0011) in the 20 μg but not in the other dose groups. In addition, both fasting (median 36%, p=0.008) and stimulated (median 19%, p=0.0156) c-peptide increased from baseline to 24 weeks in the 20 μg dose group alone. GADA levels clearly increased (p<0.001) in response to 500 μg dosage level, but not in other dosages of the medication of one embodiment of the present invention. An increase in CD4⁺CD25⁺/CD4⁺CD25⁻ T cell ratio was positively correlated with an improvement in fasting (r=0.51; p<0.005) and stimulated (r=0.34; p<0.05) c-peptide levels over 24 weeks.

The positive findings of this study of clinical safety and efficacy supports further clinical development of the present medication compositions and treatments as a therapeutic to prevent autoimmune diabetes.

It is an object of the present invention to provide a method for treating autoimmune diabetes in man.

The present invention also includes a method to monitor treatment of cell mediated inflammation from the group Rheumatoid Arthritis, Multiple Sclerosis, Graves Disease, Hashimotos, and graft rejection by measuring the number of regulatory CD4+ CD25+ T cells in vitro. For instance, this may be done by administering a drug to a patient that has or is believed to have such as disease or condition, and measuring to determine whether there is an increase in CD4+CD25+ regulatory T cells.

The present invention also includes a method to increase beta cell mass in a diabetes patient with beta cell antibodies, the method comprising administering to a human an effective amount of beta cell antigen in a pharmaceutical carrier for an effective time so as to allow regenerated beta cells improved survival such beta cells are exposed to a lower autoimmune attack than they should have been without such administration.

This method may be carried out by using beta cell antigens that include at least one component selected from the group consisting of: GAD65, GAD67, insulin, insulin-peptide, proinsulin, proinsulinpeptide, sulfatide, heat schock protein, S100 beta protein, IA-2, or any peptide, altered peptide ligand, chimeric molecule, or conjugated molecule or fragment thereof.

The administration of the beta cell antigen may be made in connection with administration of a substance capable of assisting beta cell regeneration, such as for example, at least one from the group: antiCD3-antibodies; antiCD25-antibodies; IL4; GLP-1 (Novo); NN2211 (Lilly); AC2993 (Amylin); AC2993LAR (Amylin), Betatropin (Restoragen); Glugagon-like peptide, PPAR-gamma agonist; Dual PPAR agonist; Galida (Astra-Zeneca), Mefformin and Glucophage.

It is preferred that the substance capable of assisting beta cell regeneration is administered during the period from 8 weeks prior to 8 weeks after administration of the beta cell antigen.

### BRIEF DESCRIPTION OF THE DRAWINGS

FIGS. 1A-1E are graphs of the percentage change in log GAD antibody levels (U/ml) before and at 4, 8, and 24 weeks from prime dose of the medication of the present invention, wherein the individual patients in the (A) Placebo, (B) 4 μg, (C) 20 μg, (D) 100 μg dose and (E) 500 μg dose groups are shown, in accordance with one embodiment of the invention.

FIGS. 2A-2C are graphs of the median percentage change before and at 8, 12 and 24 weeks from prime dose shown for the placebo, 4 μg, 20 μg, 100 μg and 500 μg dose groups, respectively as follows: (A) Effects on HbA_{1c}; *p=0.013, (B) Effects on fasting c-peptide/glucose; *p=0.0078 and ⁺p=0.0008, and (C) Effects on post-Sustacal® c-peptide/glucose. *p=0.0391 at 20 μg and p=0.0312 at 500 μg, in accordance with one embodiment of the invention.

FIGS. 3A and 3B are graphs of the mean change before and at 8 and 24 weeks from prime dose are shown for the placebo, 4 FIG. 20 μg, 100 μg and 500 μg dose groups in respectively as follows: (A) CD4/CD8 ratio, and (B) CD4⁺CD25⁺/CD4⁺CD25⁻ ratio, showing an increase in the ratio of CD4⁺CD25⁺/CD4⁺CD25⁻ cells over time (*p=0.012) and relation to placebo (+p=0.03), in accordance with one embodiment of the invention.

FIGS. 4A and 4B are graphs of results showing change in fasting C-peptide levels in individuals studied in accordnace with one embodiment of the method of the present invention.

FIGS. 5A-5Y are graphs of results from this a phase II cliical study in accordance with one embodiment of the present invention.

FIG. 6 is a graph describing induction of GAD65-specific regulatory T cells in NOD mice.

FIG. 7 is a chart describing the patient disposition in a Phase II trial conducted using a method in accordance with one embodiment of the present invention.

FIG. 8 is a graph describing C-peptide/glucose at 6 months, 12 months and 18 months in a Phase II trial conducted using a method in accordance with one embodiment of the present invention.

FIG. 9 is a graph describing the log of fasting C-peptide/fasting glucose at 6 months, 12 months and 18 months in a Phase II trial conducted using a method in accordance with one embodiment of the present invention.

FIG. 10 is a graph describing HbA_{1c} (%) at 6 months, 12 months and 18 months in a Phase II trial conducted using a method in accordance with one embodiment of the present invention.

FIG. 11 is a graph describing the log of GAD65Ab at 6 months, 12 months and 18 months in a Phase II trial conducted using a method in accordance with one embodiment of the present invention.

FIG. 12 is a graph describing the change in CD4⁺CD25⁺/CD4⁺CD25⁻ T cell ratio in a Phase II trial conducted using a method in accordance with one embodiment of the present invention.

FIG. 13 is a graph describing the percent of treated and control LADA patients receiving insulin in 24 Months in a Phase II trial conducted using a method in accordance with one embodiment of the present invention.

### DETAILED DESCRIPTION OF THE INVENTION

In order to treat autoimmune diabetes, the following provides an example of one embodiment that demonstrates the safe efficacy of the present invention. This is considered to be the best mode of the invention.

Trial Design

The study design was a randomised, double blind, placebo-controlled, group comparison, dose-escalation study conducted in LADA patients at the Department of Endocrinology, University Hospital MAS, Malmo, and the Department of Medicine, St. Gorans Hospital, Stockholm, Sweden. A total of 47 patients were allocated to either one of four groups receiving 4 (n=9), 20 (n=8), 100 (n=9), or 500 μg (n=8) of the medication of the present invention, or placebo (n=13). Sequential immunisation of each dosage group was conducted once the absence of safety issues were determined at lower doses. Interim safety evaluation to approve dose escalation was conducted by a separate committee 4 weeks after receipt of an injection of the medication of the present invention. In each group, nine patients were planned to receive the medication of thee present invention, and three to receive placebo. Patients treatment allocation was centrally organised by Chiltern International Ltd., Slough, Berkshire, UK.

Patients were eligible to enter the trial if they fulfilled the following entry criteria at Visit 1:1) male or female patients aged 30-70 years, 2) diagnosed with diabetes and classified with type 2 diabetes within the previous 5 years, 3) presence of GADA, 4) only requiring diabetes treatment with diet, oral hypoglycaemic agents, or both, 5) females of non child-bearing potential, 6) absence of associated serious diseases or conditions which in the opinion of the investigator would exclude the patient from the trial, and 7) patients who had given written informed consent at the screening visit.

After all patients in the 4 μg dosing group had completed visit 8 (week 8), and provided that there were no safety concerns, the next schedule of visits for the 20 μg dosing group was initiated. The same procedure was repeated for the 20, 100 and 500 μg groups. In addition, a maximum of two additional booster injections was allowed in the 500 μg dose group alone depending on the patients GADA response. The criterion for additional booster injections in the top dose group were a GADA titre that remained unchanged at week 8 (defined as less than a doubling of titre prior to receipt of the first dose) and the absence of safety concerns for that patient. A final booster injection (i.e. 4^{th} administration) was performed if there was still no change in GADA titre four weeks after the initial booster injection and if no safety concerns were apparent. All patients stayed at the hospital overnight for safety observation after each injection.

During the 24-week study period, each patient was followed at regular intervals as outpatients with a total of 10 study visits during which assessment of immunological markers, diabetic status, fasting lipids, haematological and biochemical parameters, as well as physical examinations, reporting of concomitant medication and adverse effects were performed. GADA and IA-2A were determined as previously described.¹⁵ Our laboratory is number 156 in the Diabetes Antibody Standardisation Program (DASP) for GADA and IA-2A.¹⁶ Diabetes status assessment included fasting glucose, fasting and 2-hr Sustacal® stimulated c-peptide, and long-term metabolic control assessed by HbA_{1c}. Blood samples for haematology were analysed for haemoglobin, red cell count (including MCV and MCHC), haematrocrit ratio (PCV), white cell count, differential white cell count and platelets, and biochemical parameters included analysis of plasma levels of glucose, c-peptide, HbA_{1c}, urea, creatinine, phosphorus, total bilirubin, alkaline phosphatase, alanine transferase, glutamyl transferase, lactic dehydrogenase, amylase, albumin, c-reactive protein, total protein and fasting lipid and lipoproteins. Clinical neurological assessment and EMG were performed at baseline and after 6 months to detect any adverse effects on the neuromuscular system.^{17,18}

Patients

Patient characteristics for patients receiving placebo and for the four dose groups at baseline are given in Table 1. All patients remained in the study for 24 weeks. One patient given placebo started insulin treatment at 12 weeks along with one patient in each of the 100 μg and 500 μg groups, who started insulin treatment after 12 but before 24 weeks. At week 24, only fasting c-peptide was available from these patients. For technical reasons, c-peptide were not available at week 24 from one patient in the placebo (fasting) and another in the 100 μg (stimulated) group, respectively. Three patients did not complete the study for personal reasons, one each in the 4, 20 and 100 μg groups.

Test Substances

Sterile, pre-filled vials of the medication of the present invention were provided by Diamyd Therapeutics AB, Stockholm, Sweden for clinical trial use. The unmodified recombinant form of human GAD65 (bulk rhGAD65) was formulated with aluminium hydroxide, such as that sold under the trademark Alhydrogel®. The bulk rhGAD65 was manufactured using baculovirus/insect cell expression of the cDNA for hGAD65.¹⁹ Both manufacture of the bulk rhGAD65 and that of the medication of the present invention were performed under strict conditions of current Good Manufacturing Practice. Each vial contained a sterile formulation of either 4, 20, 100, or 500 μg of the medication of the present invention having a constant amount of Alhydrogel®. Coded vials containing an identical amount of Alhydrogel® alone were used as placebo.

Flow Cytometric Analysis

Flow cytometric analysis of lymphocyte subsets was conducted using standard techniques. Whole blood was stained at room temperature for 20-30 minutes with monoclonal antibodies against CD3, CD4, CD8, CD25, CD19, CD56 and CD16 (all from Becton Dickinson, Calif., USA). Erythrocytes were lysed using a FACS® tradeamrk lysis buffer containing paraformaldehyde (Becton Dickinson, CALIFORNIA, USA). Thereafter, cells were washed, resuspended in staining PBS buffer containing 0.5% bovine serum albumin and 2 mM EDTA (pH 7.4) and analysed within 24 hrs using a FACSCalibur® (Becton D ickinson, Franklin Lakes, N.J., USA) and CellQuest® software (Becton Dickinson). Between 10,000 and 50,000 events were acquired and the absolute number of each subset was calculated by multiplying the percentage of cells by the total lymphocyte count obtained at the hospital clinical laboratory for the same blood sample. Isotype-matched control antibodies (IgG2a FITC) were used to set the dot plot quadrant and calculate the percent of CD4⁺CD25⁺ lymphocyte population.

Statistical Analysis

All graphs and analyses were performed using Splus6.1 (from Insighfful Corp., Seattle, Wash.). Two-sample t-test was used for differences in means between variables normally or symmetrically distributed. When distributions were not symmetric, medians were used as a summary measure and the corresponding Wilcoxon two-sample test was used to evaluate statistical significances. Change in plasma glucose, c-peptide and HbA_{1c} for each subject was presented as percent change in level from baseline. Change in T cell counts was summarised as change in ratio totals. Multiple linear regression was used to test whether differences observed in the univariate analyses remained after adjusting for variables such as age, BMI and gender as well as GADA and IA-2A. C-peptide was log transformed to assure normality and constant variance assumption. The 4 μg dose group was considered as having no effect and was therefore combined with placebo as controls and compared to a treatment group. Spearman's correlation coefficient tested the association between change in c-peptide levels and change in CD25⁺/CD25⁻ composition within CD4⁺ T cells. P-values less than 0.05 were considered significant.

Results

Study-Related Adverse Events

There were 32/47 patients (68%) with 51 adverse events (AE). The majority experienced influenza-like symptoms, with nasopharyngitis being the most common AE. Prior to study completion and unblinding three AEs were judged to be probably related to the trial treatment, i.e. vitiligo (later found to be in the placebo group), mild leukocytosis (100 μg dose group) and a mild inflammation at the injection site on the left arm (500 μg dose group). As a precautionary measure the patient with vitiligo was withdrawn from the trial two weeks after the first injection. However, leukocytosis and inflammation were completely resolved in the two other patients and no separate treatment was required. There were no severe AEs or deaths during the trial.

Injection site reactions were absent at the majority of visits. All reactions were mild and most, in particular tenderness, occurred primarily on the day of the first injection (day 1) and on the day of the second injection (week 4).

Mean haematology and biochemistry parameters were within normal limits in each treatment group at most visits. There were no significant changes from baseline (day 1) in any of the parameters except for two patients had abnormal clinically significant laboratory results; one patient (20 μg dose group) having raised liver enzymes and another (placebo dose group) having raised blood iron levels, later diagnosed with haemochromatosis.

There were no differences between treatment groups in blood pressure or pulse rate, no deterioration in neurological assessment, muscle tone and spasms, and no abnormal EMG assessments.

GAD65 Autoantibody (GADA) Levels

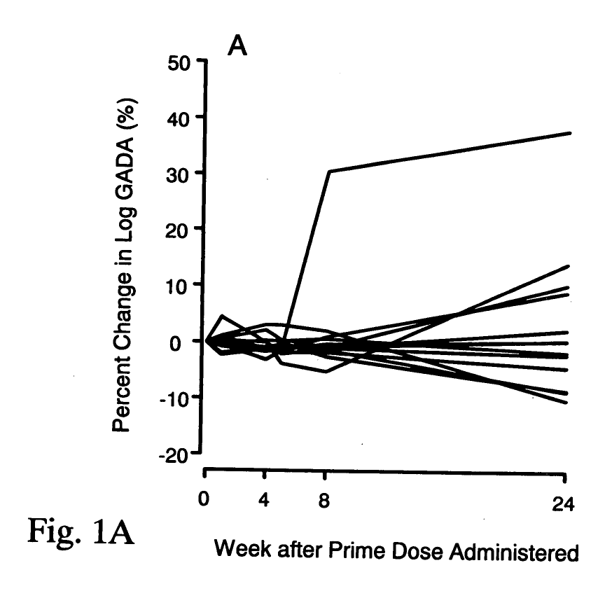

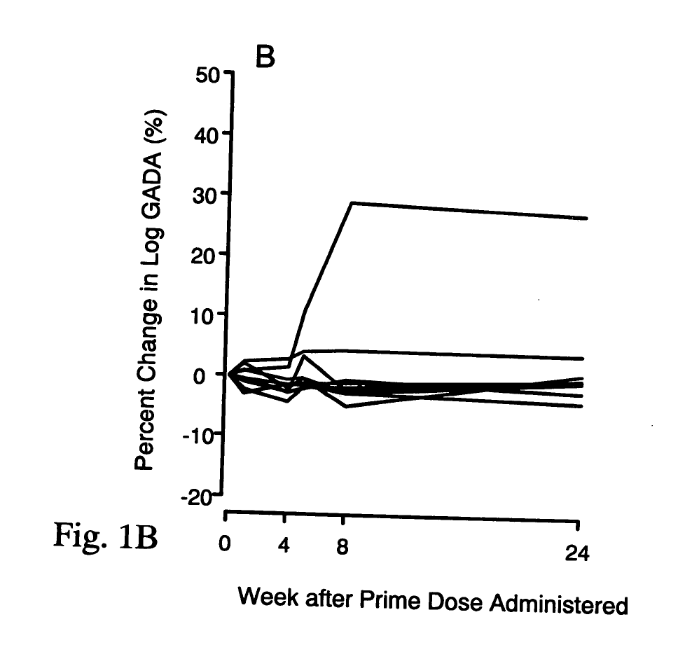

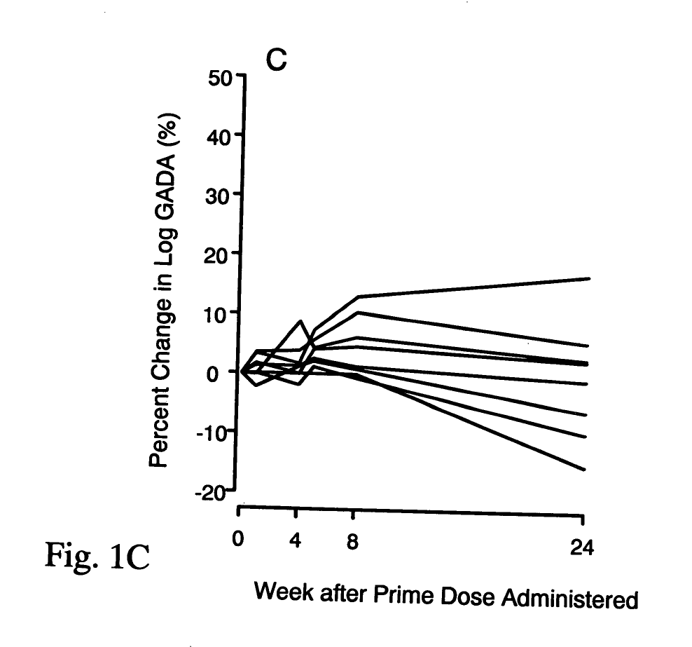

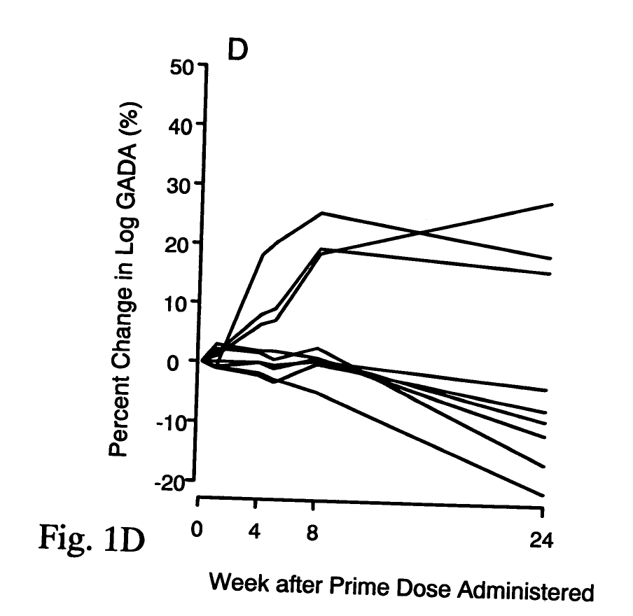

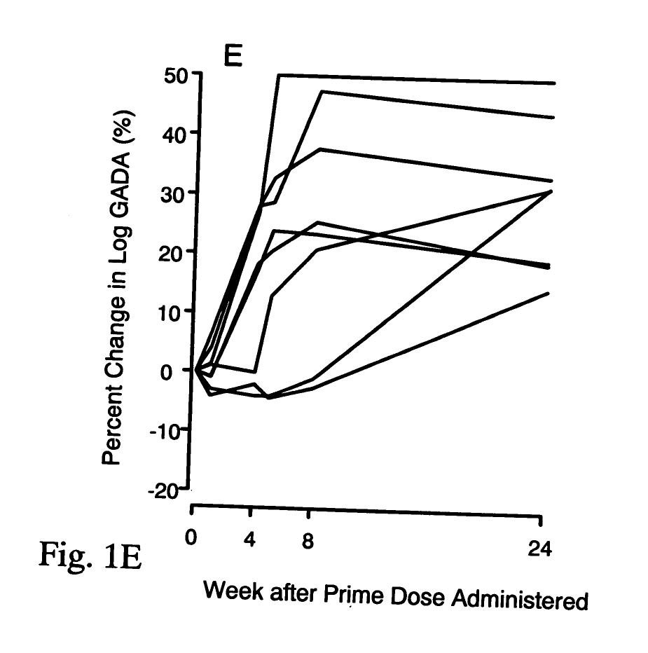

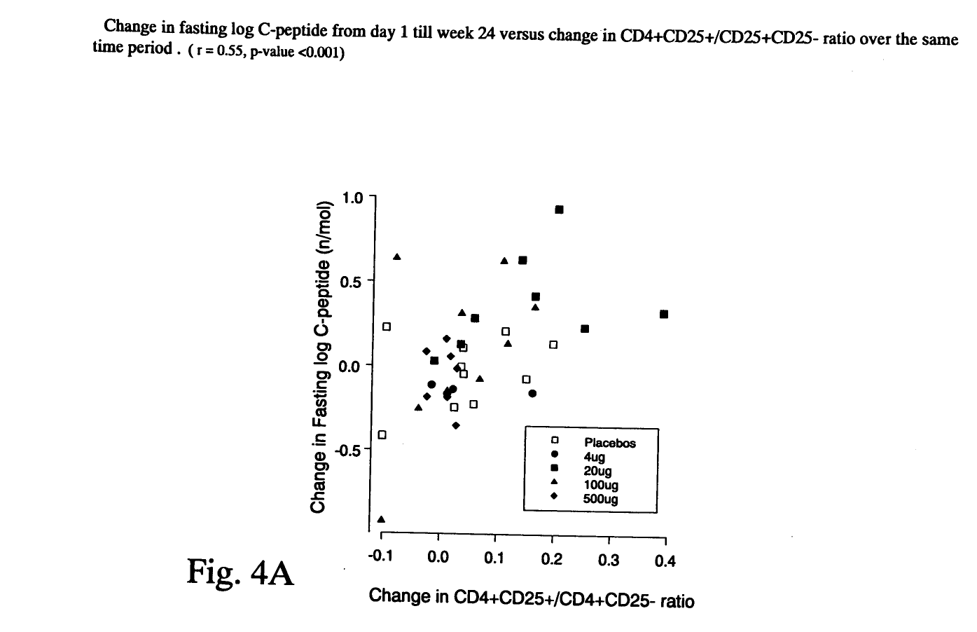

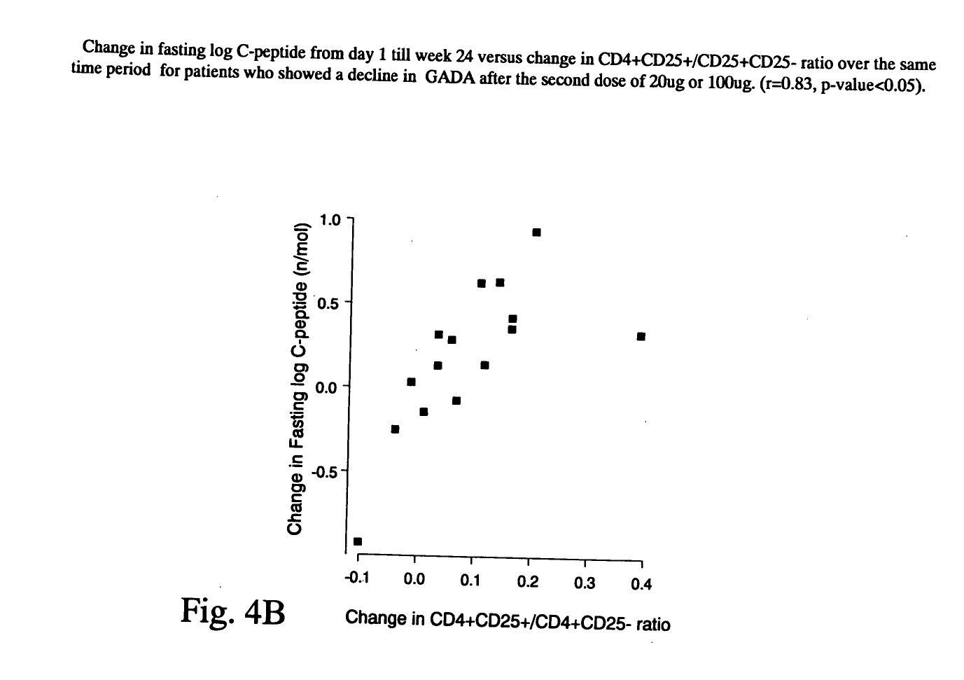

The percentage change in GADA levels from day 1 for all patients and all groups indicate no change after 1, 4 (when the boost injection was given), 8 and 24 weeks in the placebo (FIG. 1A) and 4 μg (FIG. 1B) groups. Levels in the 20 μg dose group did not change during the first 4 weeks; however, between 8 and 24 weeks 7/8 (88%) patients showed a decline (FIG. 1C). Similarly, in the 100 μg dose group there was a decline in 8/9 (89%) patients despite an increase in three patients between 4 and 24 weeks (FIG. 1D). In these two groups the number of patients with a decline (15/17) was different from the placebo group (6/13; p<0.05). All eight patients in the 500 μg group developed either an early increase before week 4 or a gradual increase after additional boost injections during the 24 weeks (FIG. 1E).

Blood Glucose and HbA_{1c} Levels

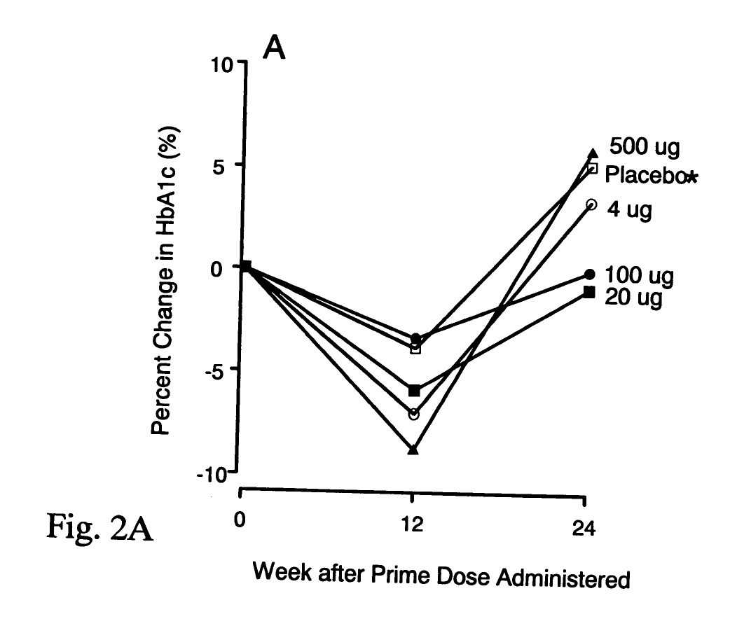

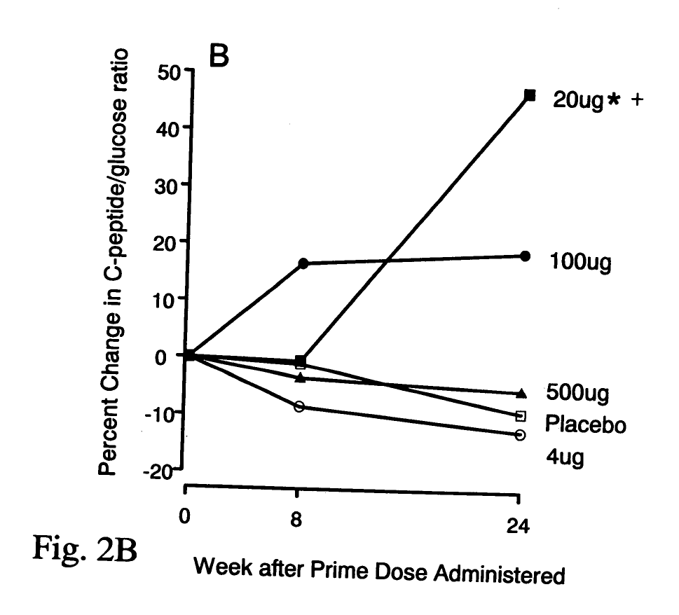

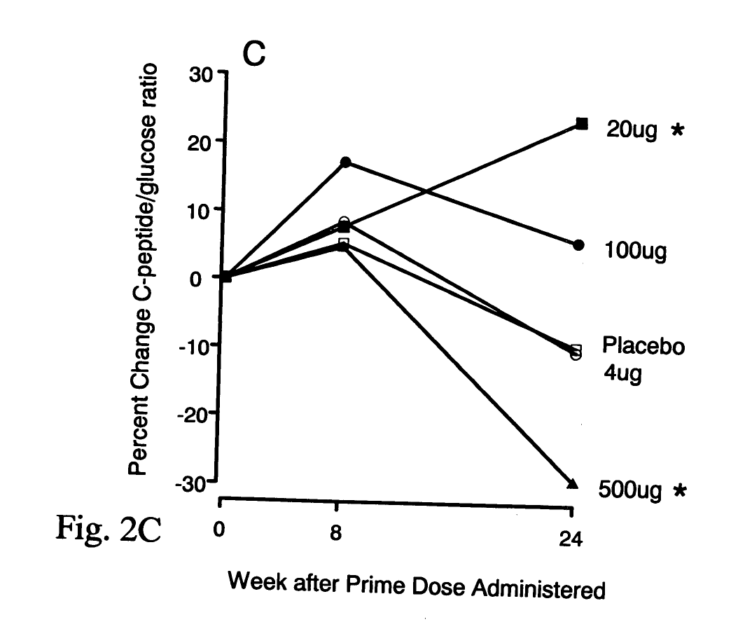

There were no changes in fasting blood glucose during the first 24 weeks. Within the placebo group, however, there was considerable variability in percentage change. An increase in HbA_{1c} levels (p=0.01) was found in the placebo but not in the other dose-groups (FIG. 2A).

Fasting and Sstimulated C-Peptide

The groups did not differ in fasting c-peptide levels at visit 2 (Table 1). However, the 20 μg dose group showed an increase in fasting c-peptide compared to placebo (p=0.0011) as well as an increase in both fasting (36%, p=0.008) and stimulated (19%, p=0.0156) c-peptide at 24 weeks. This effect was also evident when the data were calculated as a function of c-peptide/glucose (FIGS. 2B and C) and compared to both baseline (47% increase, p=0.0078) and to placebo (p=0.0008) (FIG. 2B). An increase in levels compared to baseline within the 20 μg group was also recorded in stimulated c-peptide/glucose (24% increase, p=0.0391) (FIG. 2C). A decline in stimulated c-peptide/glucose levels was apparent in the 500 μg dose group (p=0.03). Patients given placebo or the 4 μg dose had almost identical changes in c-peptide/glucose ratio during the 24 weeks follow-up (FIG. 2C).

Blood Lymphocytes and T Cell Subsets

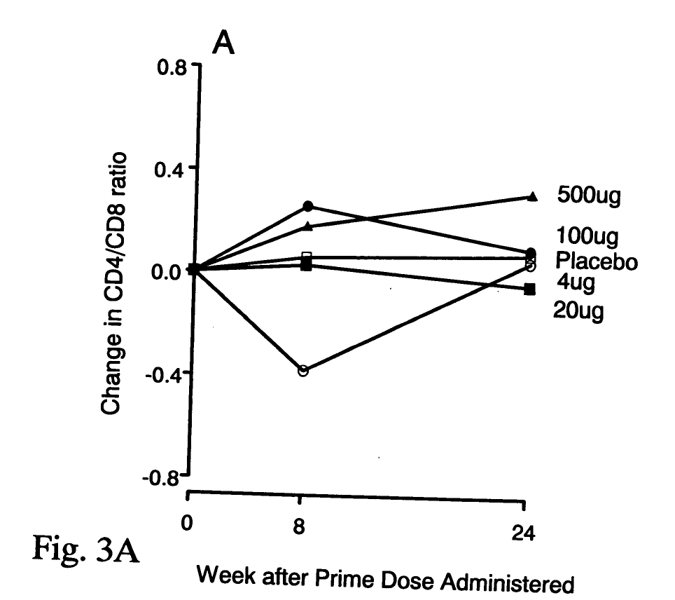

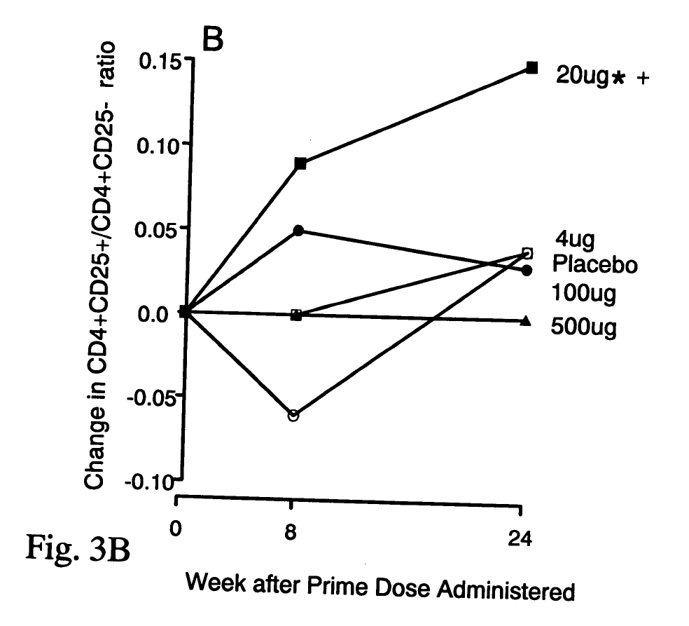

At day 1 and during the 24 weeks of observation no differences between the groups were observed in total CD3⁺, CD4⁺, or CD8⁺ T cells, B cells, NK cells or T cells with NK cell markers or in the CD4⁺/CD8⁺ T cell ratio (data not shown). In contrast, we observed that the ratio of CD4⁺CD25⁺/CD4⁺CD25⁻ T cells increased (p=0.012) over time in the 20 μg group, but not in the other groups (FIG. 3). This change was also different from the placebo group (p=0.03, FIG. 3). There was a positive correlation in all medication-treated patients between change in CD4⁺CD25⁺/CD4⁺CD25⁻ T cell ratio and change in fasting c-peptide (r=0.51; p<0.005) as well as in post-Sustacal® c-peptide (r=0.34; p<0.05), demonstrating that patients exhibiting an increase in c-peptide also had an increase in CD4⁺CD25⁺/CD4⁺CD25⁻ T cell ratio.

Multiple Linear Regressions

Changes in HbA_{1c}, fasting glucose, fasting and stimulated log c-peptide in the control (n=21) were compared to the treatment (n=24) group. Unadjusted analyses confirmed the increase compared to controls in fasting log c-peptide (+0.208, p=0.025) and a decrease in HbA_{1c} (−0.603, p=0.024). The effect on fasting log c-peptide remained after adjusting separately for age, gender, BMI, HbA_{1c} at baseline as well as GADA and IA-2A.

Findings and Discussion

The results of the study support the clinical safety of subcutaneous administration of alum-formulated recombinant human GAD65 as well as its ability to increase c-peptide levels and affect the CD4⁺CD25⁺ T cell subset in peripheral blood.

Cutaneous reactions to the treatment of the present invention were minor and of no clinical significance. These findings support the results of a phase I clinical study demonstrating that subcutaneous administration of rhGAD65 was well-tolerated. In the study of the present invention, inclusion of a patient group receiving a provisional no effect dose level (4 μg) was intended to provide clinical outcomes indistinguishable from placebo and from which dose escalation could be safely performed. The additional booster injections with 500 μg of the medication of the present invention were intended to maximise the likelihood of immunomodulation resulting therefrom (apparent as a doubling in GADA). Three patients in the 500 μg dose group received additional injections. Of these, two patients received one additional injection (a total of 3 injections) and one patient received two additional injections (a total of 4 injections). No study-related adverse events were observed in any of the patients in the 500 μg group despite a more than two-fold increase in GADA. The stimulatory effects of 20 μg of the medication of the present invention on both fasting and stimulated (post-Sustacal®) c-peptide was observed when the patients were either compared to their change from baseline or when compared to the placebo group. It is possible that the c-peptide response to the medication of the present invention is dependent on both the dose and the individual since some of the patients receiving 100 μg of the medication also showed an increase in fasting c-peptide levels. It is also noted that patients receiving 500 μg of the medication tended to show a decline in fasting c-peptide, indicating a possible dose dependent effect. The effects of 20 μg of the medication on both fasting and stimulated c-peptide were statistically significant whether or not these were calculated as c-peptide/glucose ratio or c-peptide levels alone. As the increase in c-peptide levels in the 20 μg medication dose group was not associated with a corresponding decrease in HbA_{1c} levels, the increase in c-peptide was probably not an effect of decreased glucose toxicity on the beta cell. However, the positive correlation of CD4⁺CD25⁺ T cells to the increase in c-peptide in the 20 μg dose group suggests an immunomodulatory mechanism. Further evidence for immunomodulation is provided by the median decline in log GADA after 20 μg (−0.17 U/ml) and 100 μg (−0.33 U/ml) of the medication, indicating a possible suppressive effect on GADA production. This suppressive effect may be explained by an increase in CD4⁺CD25⁺ T cells since the CD4⁺/CD25⁺/CD4⁺CD25⁻ ratio among patients in either the 20 or 100 μg dose groups showing a decline in GADA correlated to an increase in fasting c-peptide (r²=0.83; p<0.005). These results also support the increase in c-peptide being related to a quantitative increase in CD4⁺CD25⁺ T cells.

CD4⁺CD25⁺ T cells are regarded as regulatory T cells^{20,21} and in experimental animals their presence confer inhibition of autoimmunity.²² Although not limited by the mechanism of the invention, several mechanisms may explain the positive effect on c-peptide levels in patients receiving 20 μg (and in some receiving 100 μg). First, 20 μg of the medication may induce specific T cells recognising immunodominant GAD65 epitopes. These GAD65-specific regulatory T cells would down-regulate existing GAD65 autoreactive T cells and thereby preserve c-peptide. As a second possibility, non-antigen specific CD4⁺CD25⁺ T cells may be induced in numbers sufficient to allow their detection in peripheral blood. As such, CD4⁺CD25⁺ T cells were shown to be immuno-suppressive²⁴ their greater number could possibly down-regulate self-reactive T cells, thereby inhibiting T-cell-mediated beta cell killing. As no changes in other lymphocyte subsets were found, the present treatment could be considered immunologicaly safe with regard to its clinical impact on peripheral lymphocytes.

The effect of the medication of the present invention may be highly dose dependent, as is well-known in specific immunomodulation of certain allergies. In addition to T cell subset immunomodulation, administration of the medication may also impact on B cell activity, since GADA levels in the 20 μg and all but three patients in the 100 μg dose groups tended to decrease over time. Indeed, it cannot be excluded that therapeutic activity also extends to the 100 μg dose level, since some of these patients showed an increase in fasting c-peptide that was associated with an increase in CD4⁺CD25⁺ ratio and a decrease in GADA. Although it is possible that the immune response to administration of low levels of the medication involves a shift towards less aggressive cytokines produced by certain T cell subsets, the effect of 20 μg dose does not suggest a shift from a Th1 to a Th2 response, as this would otherwise be indicated by an increase in GADA.¹³ Rather, because of their reported regulatory activity^{20,21} and their demonstrated decrease in conditions of autoimmunity,^{22,23} the induction of CD4⁺CD25⁺ T cells by low doses of the medication suggest a novel mechanism by which autoimmunity is down-regulated.

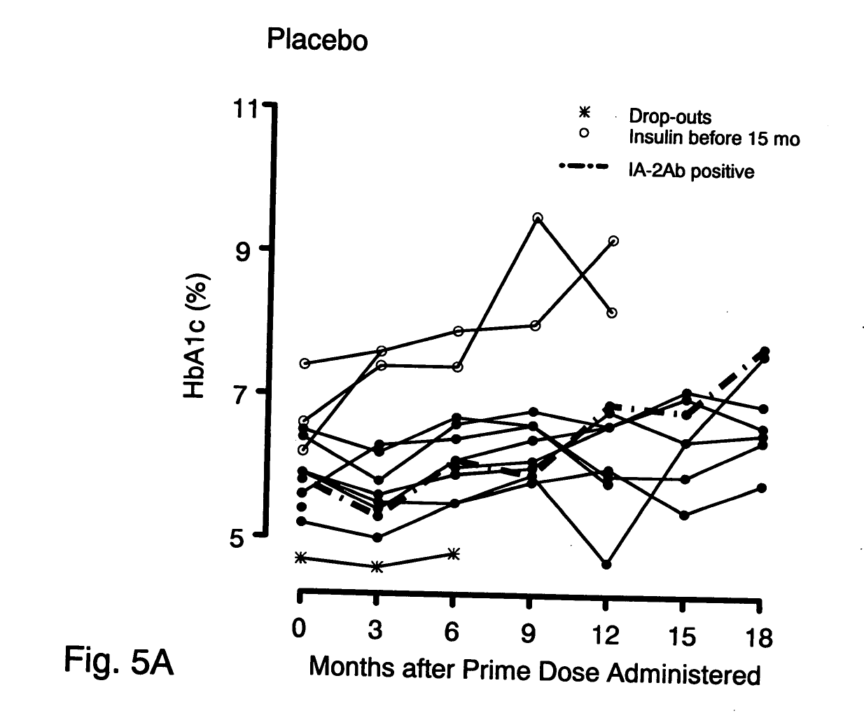

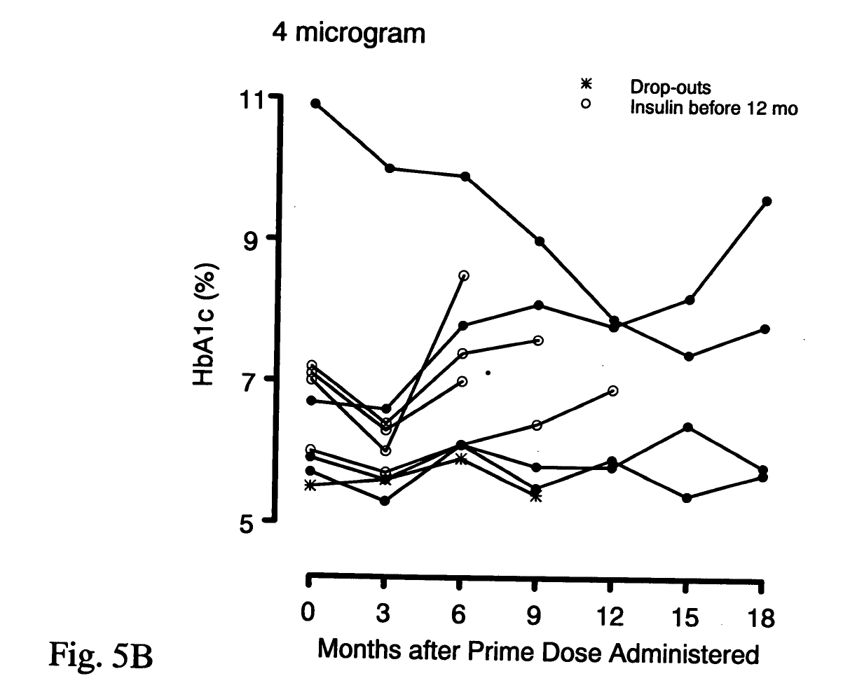

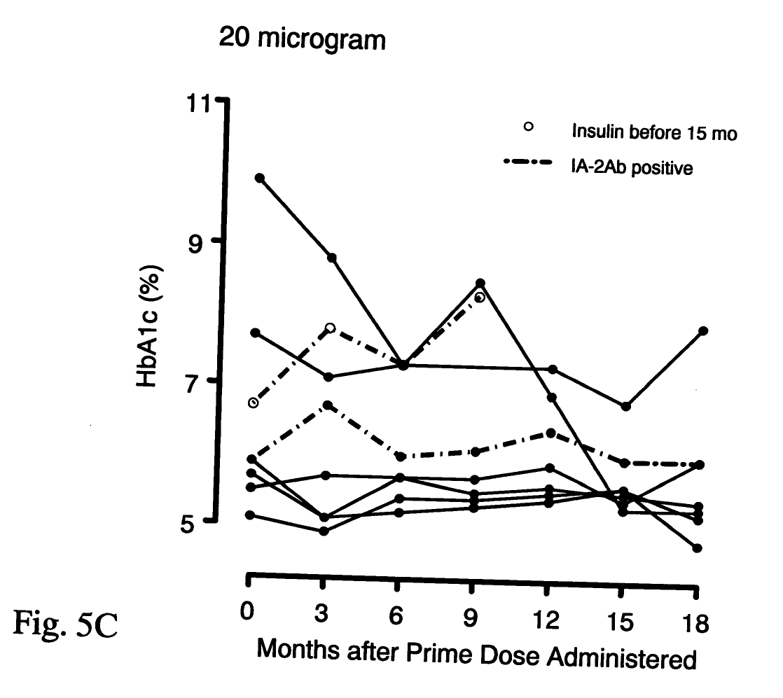

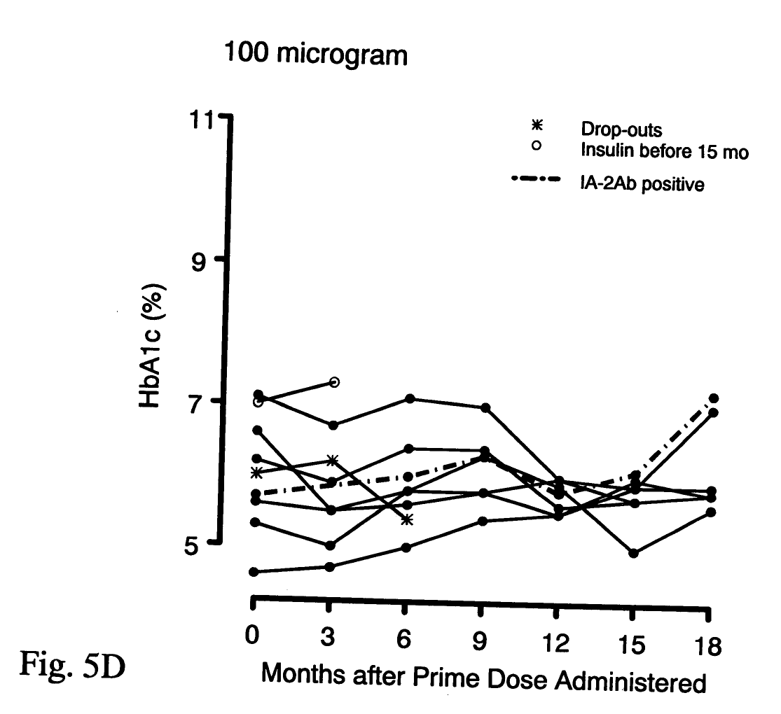

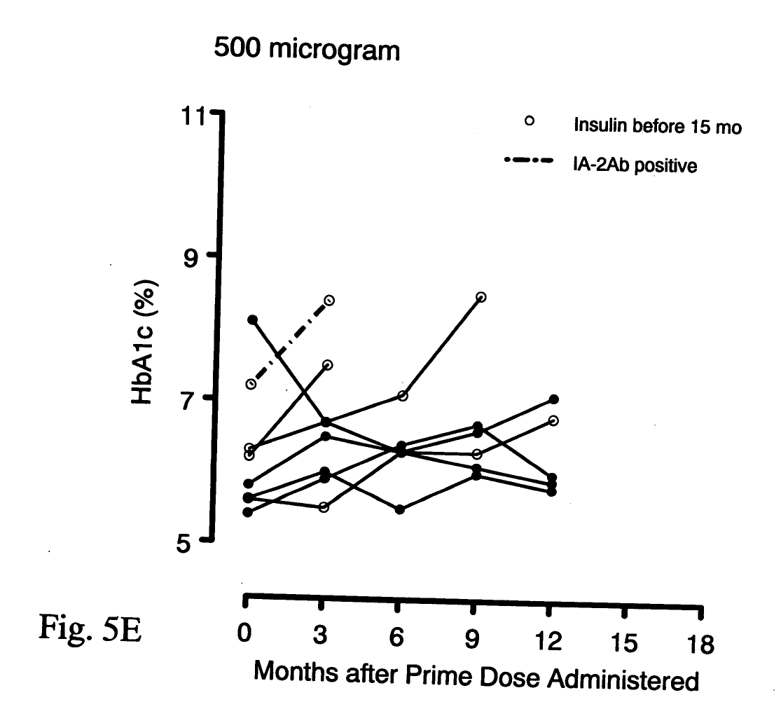

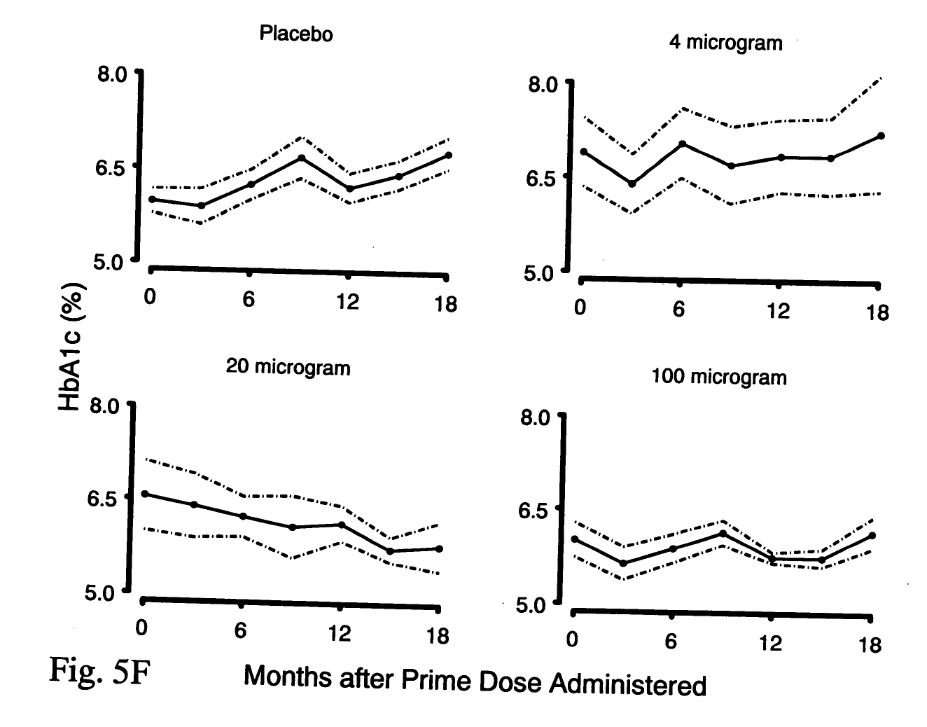

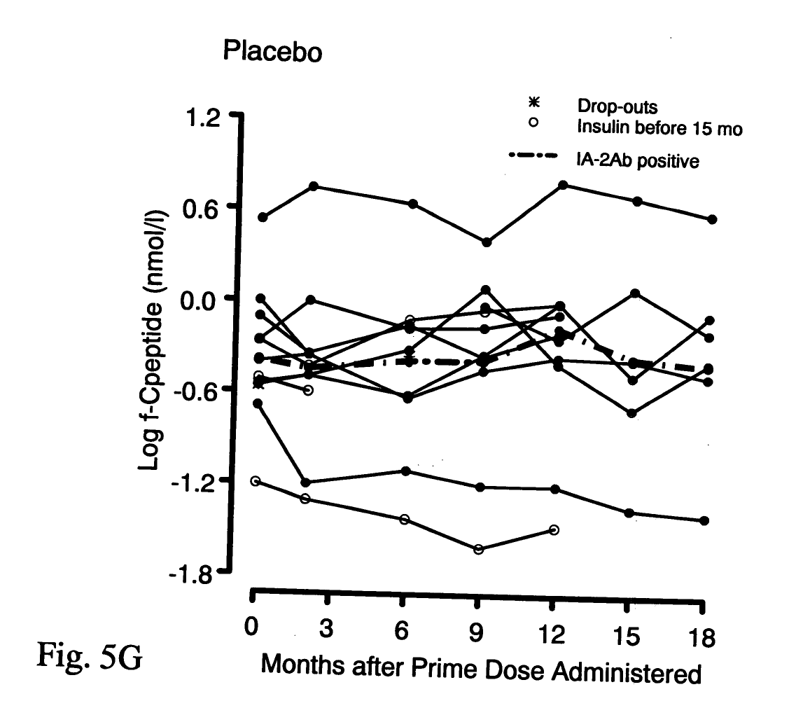

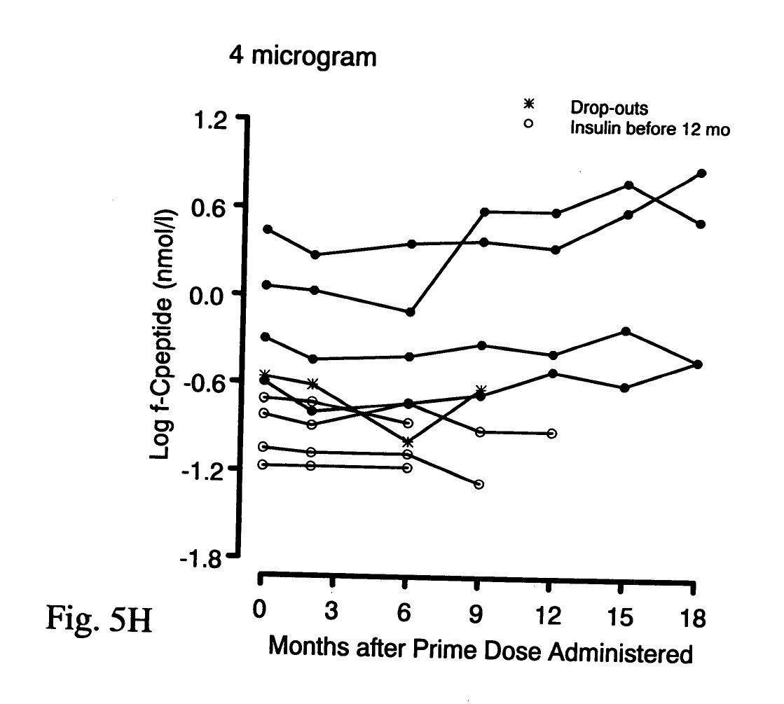

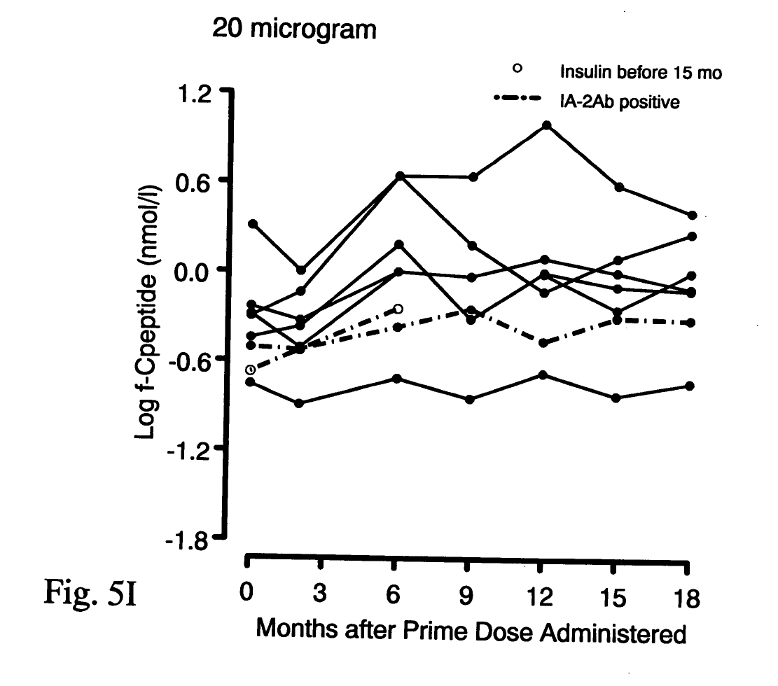

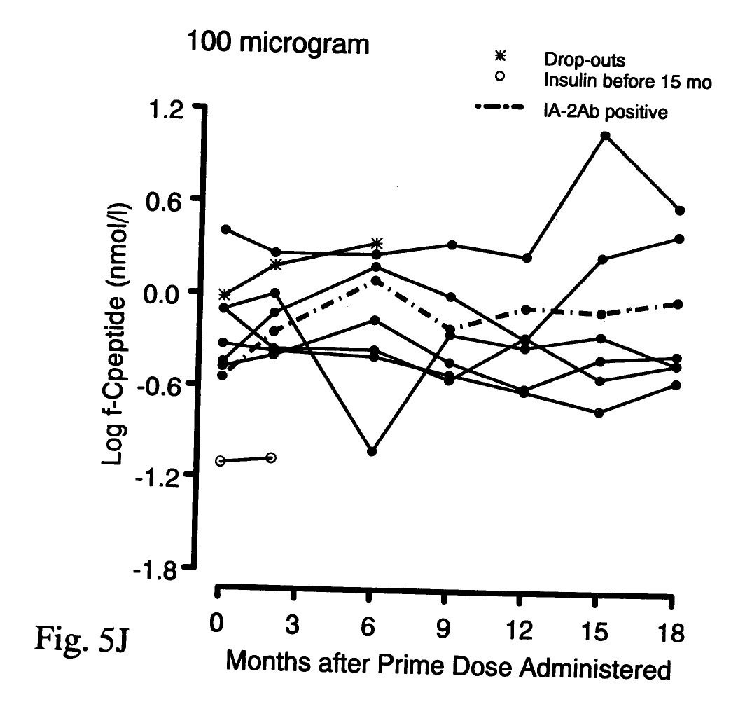

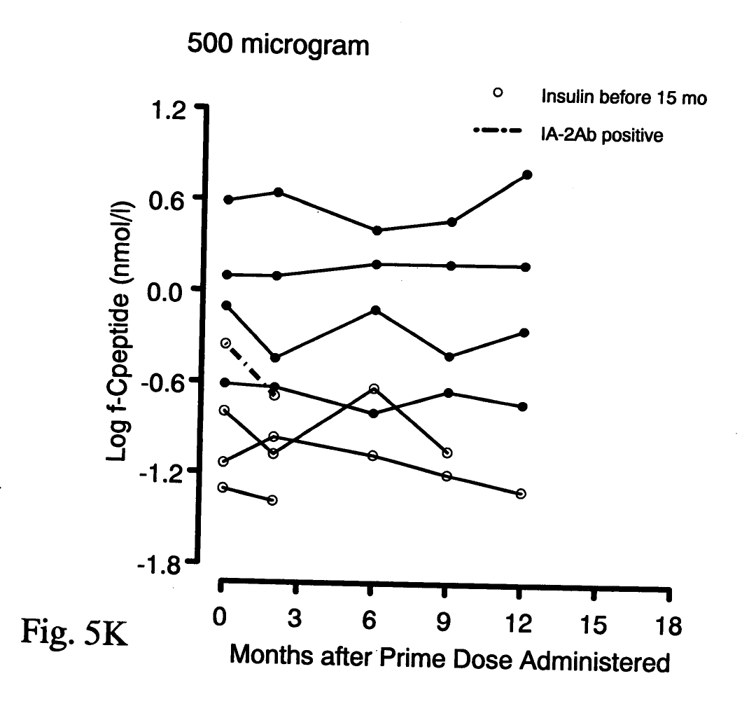

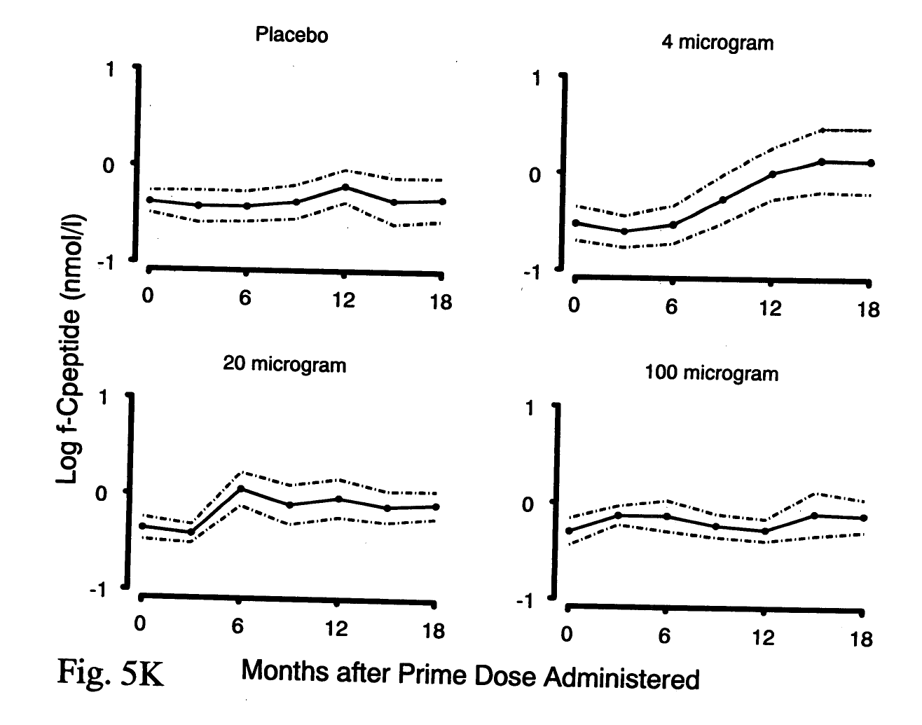

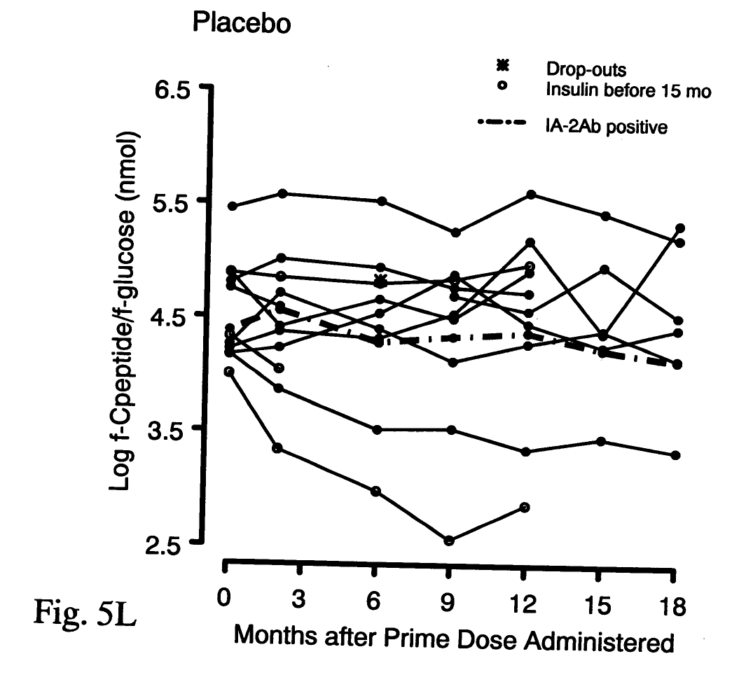

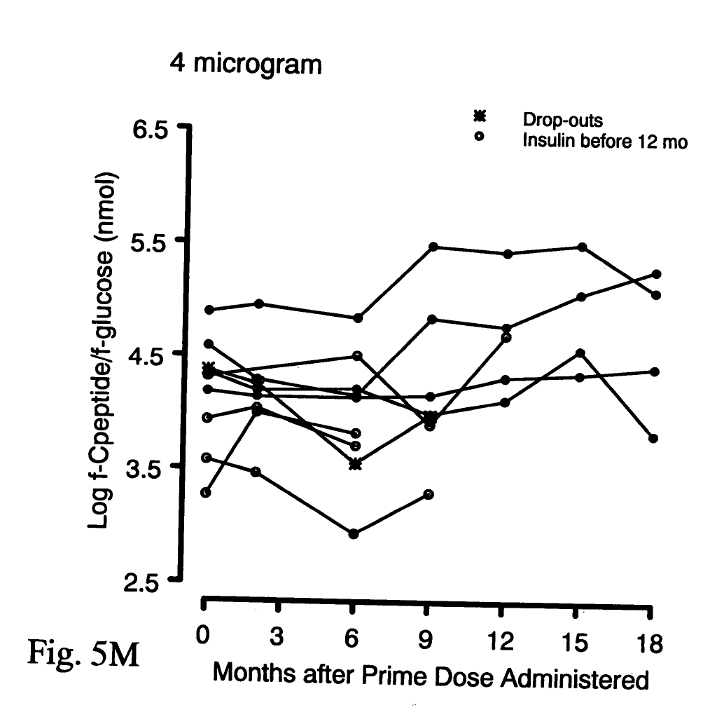

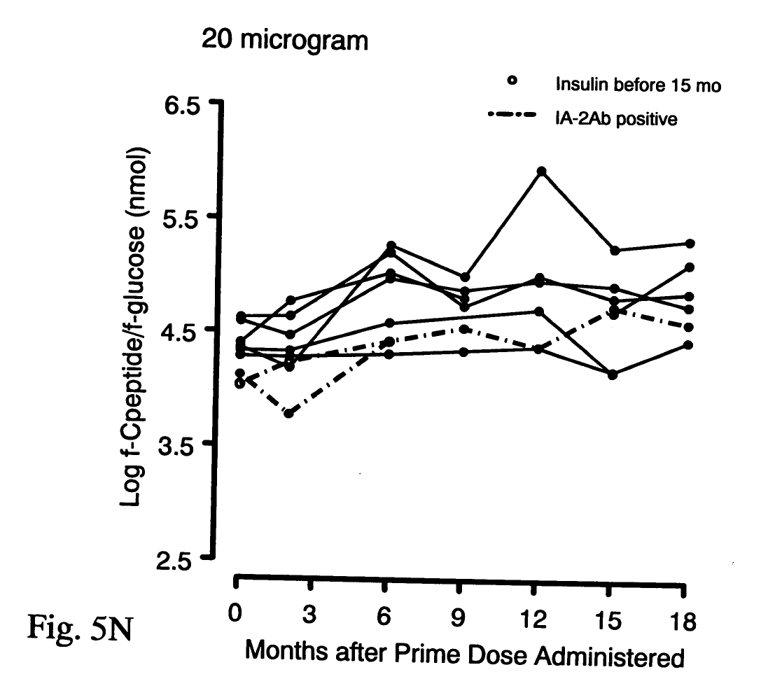

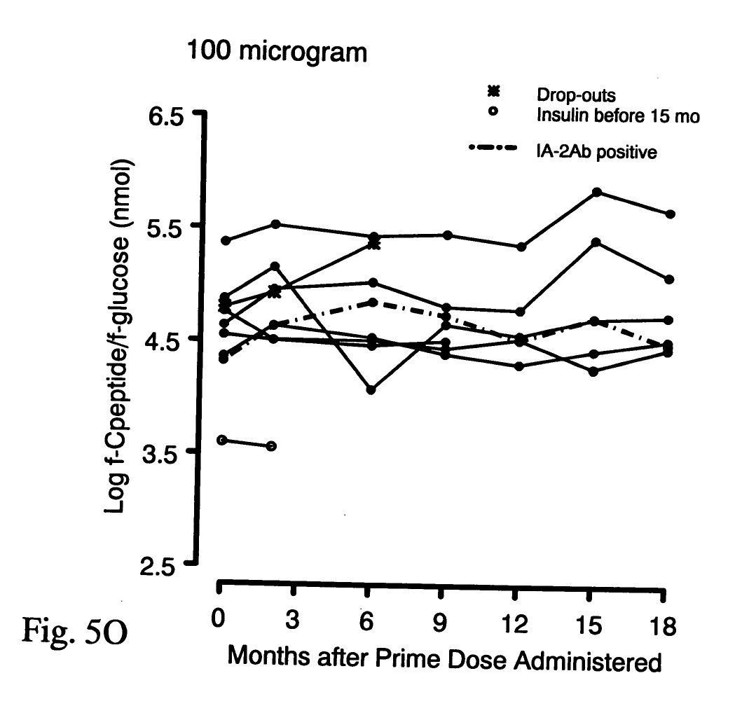

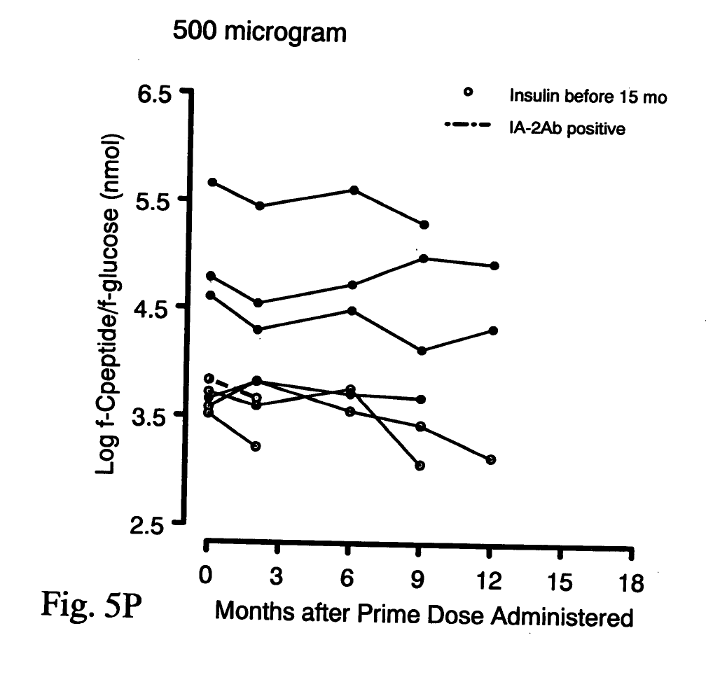

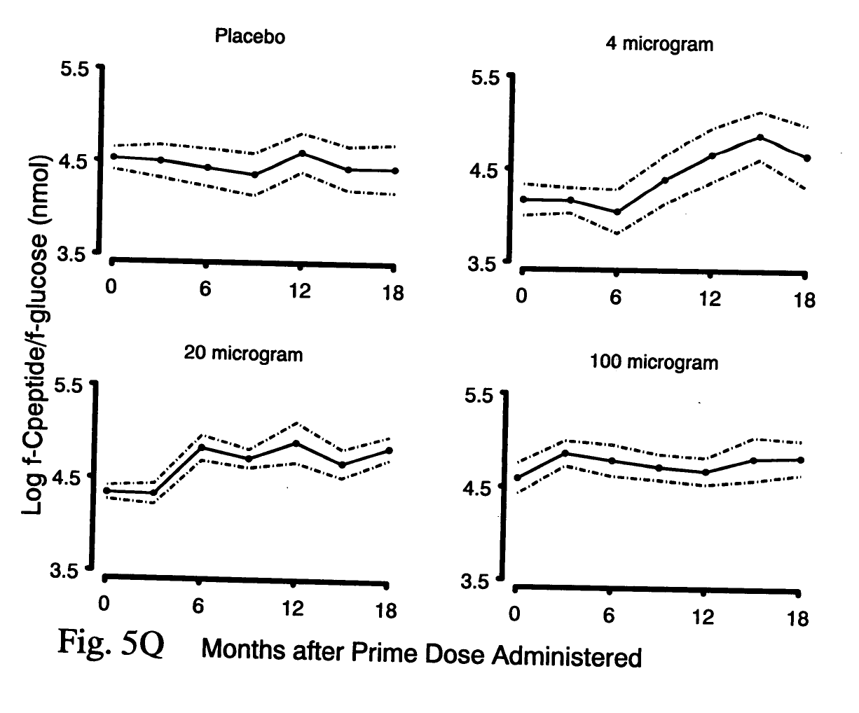

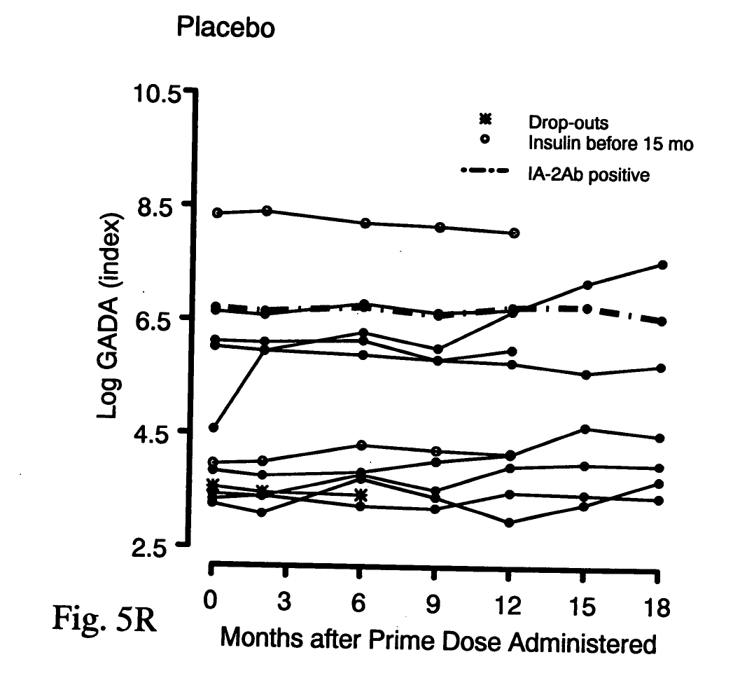

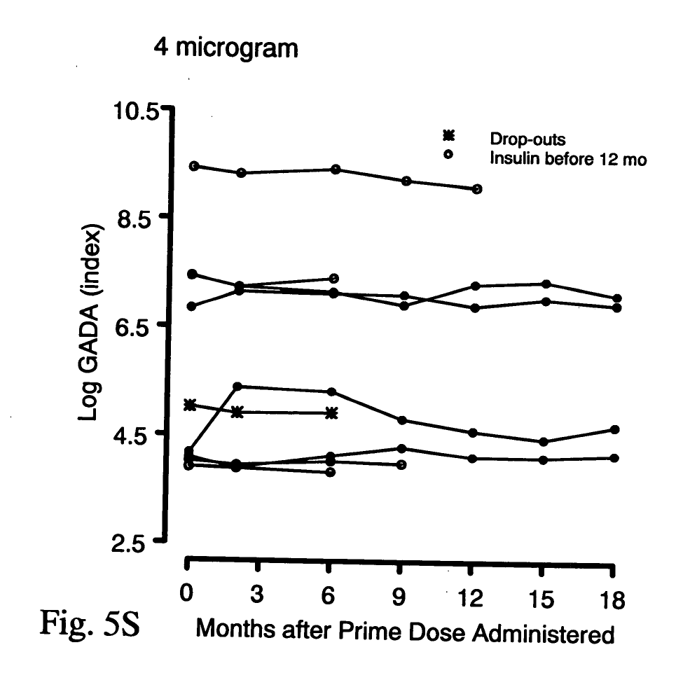

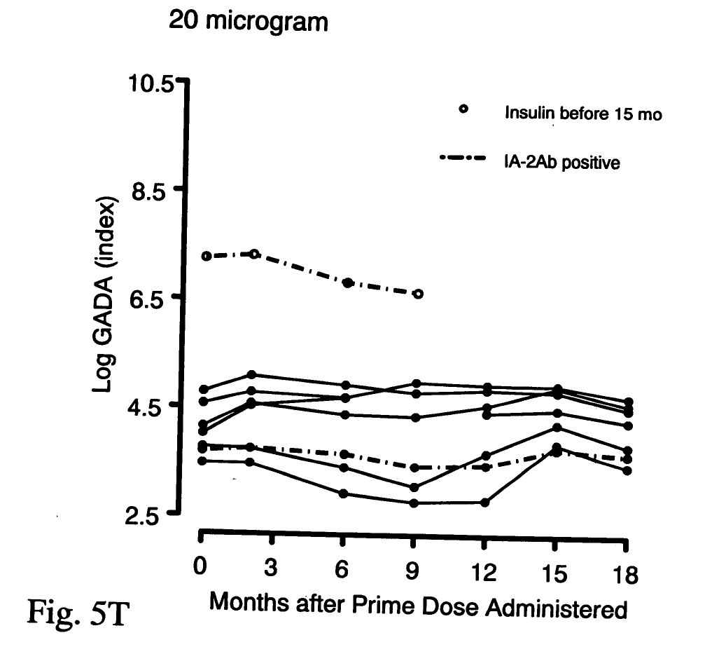

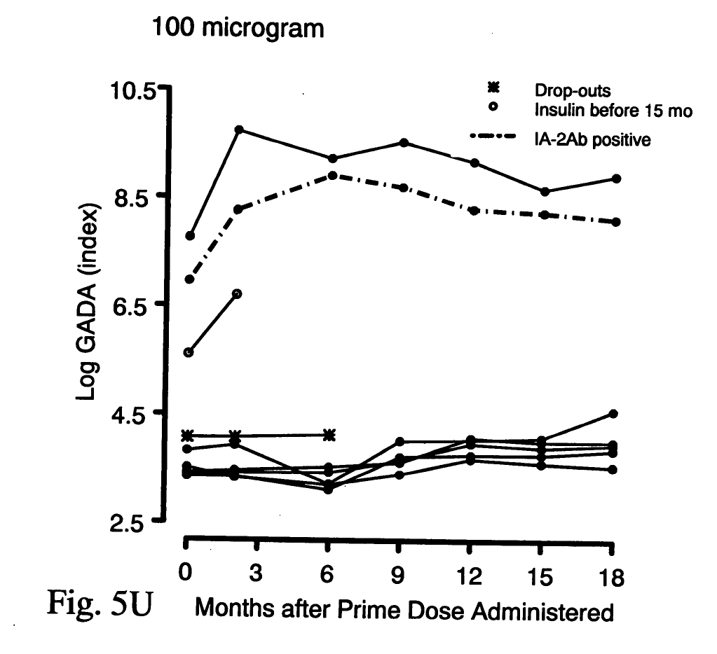

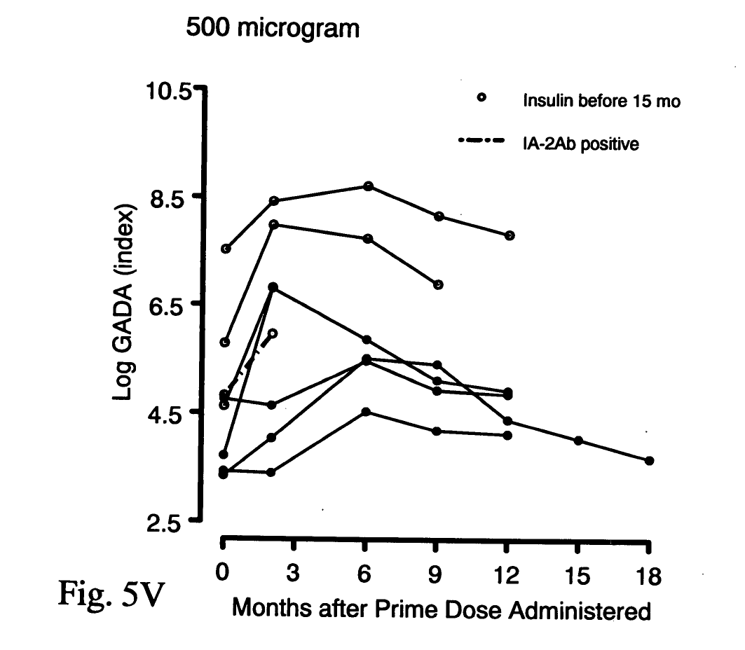

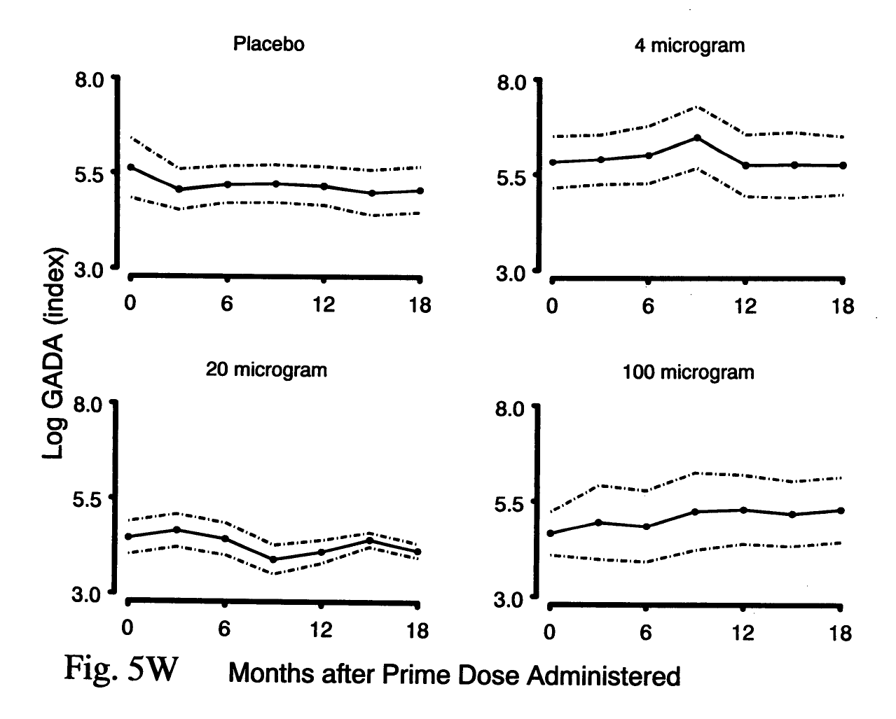

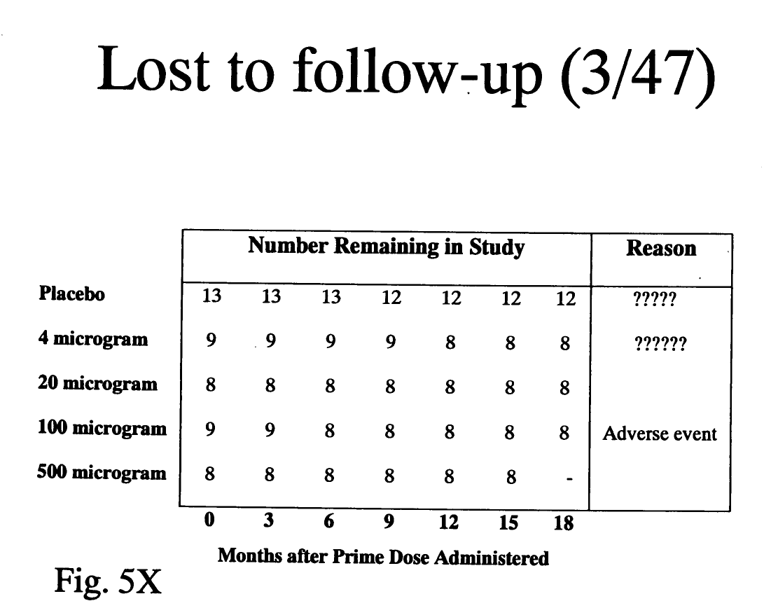

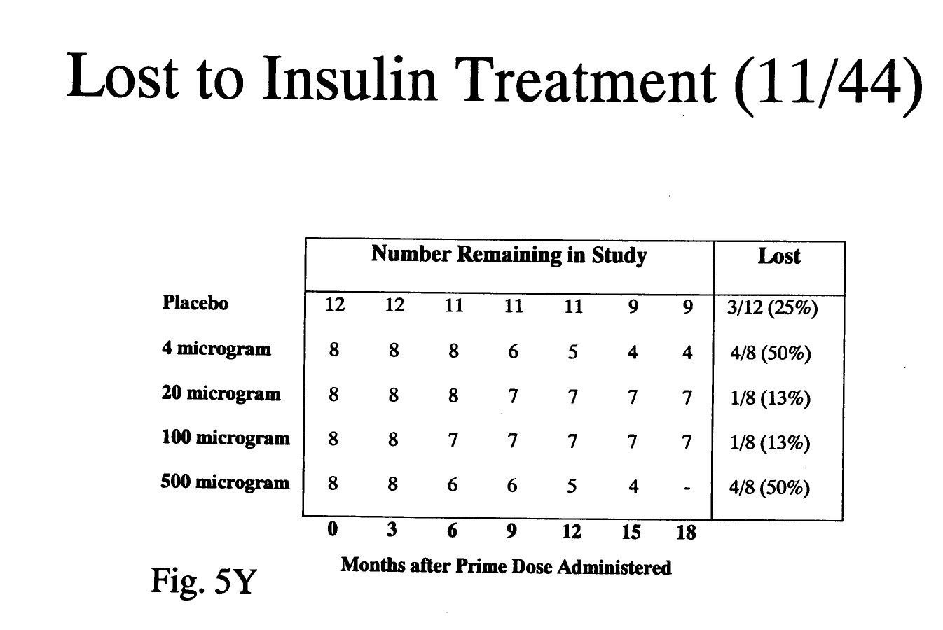

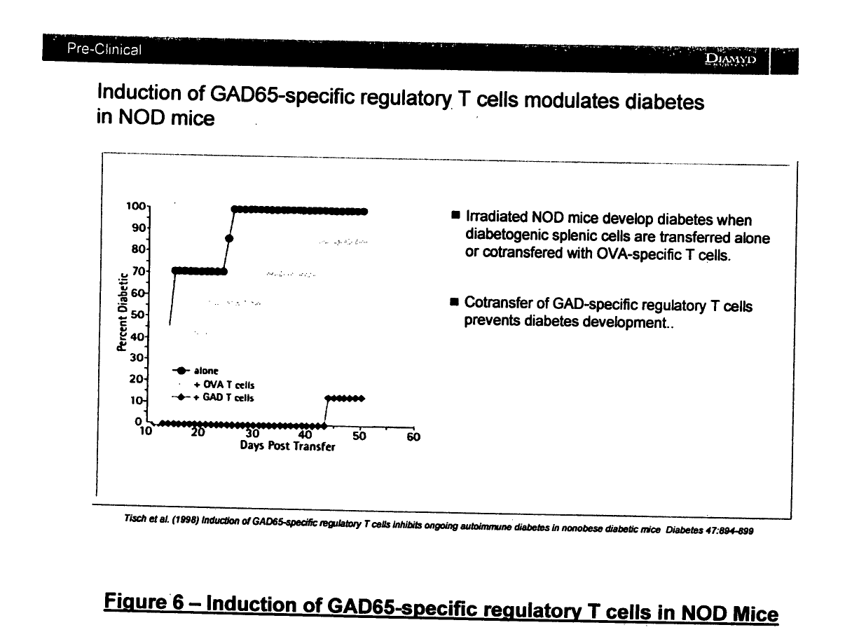

GAD65 immunomodulation in GAD65 autoantibody positive type 2 diabetes patients was studied to determine the immunomodulation of GAD-specific autoimmunity as a potential therapy of type 1 diabetes. A phase II clinical trial on 47 GAD65 autoantibody positive type 2 diabetes patients previously reported no severe adverse events after six months. The primary aim was to examine if the proposed invention was still clinically safe after 12 months. A secondary goal was to determine whether GAD65 administration prevents progression to insulin dependency after one year. Patients with LADA received placebo or 4, 20, 100 or 500 μg subcutaneously at weeks 1 and 4 in a randomised, double blind, group comparison dose-escalation study. Beta-cell function tests were performed at 2, 6, 9 and 12 months. The 4 μg was a non-effect dose group and therefore combined with placebo to form a control group. None of the patients showed significant study related adverse events and there was no sign of beta-cell collapse. While 4 of 47 received insulin before 6 months, 7 of 43 patients became insulin dependent between 6 and 12 months, and an additional 3 of 43 dropped out for unrelated reasons. Of these insulin-dependent patients 6 of 19 where controls, 1 of 8 was given 20 μg, 0 of 7 100 μg and 0 of 6 500 μg doses (p<0.05). Of the remaining 33 patients followed for the entire year, HbA_{1c} level at the start of the study in treatment patients correlated with a decline over 12 months (n=20; r=0.84; p<0.0005), but no such association was seen in the control group (n=13; r=0.32; p=NS). Fasting and stimulated C-peptide levels, which increased in the 20 μg dose group after first 6 months, remained unchanged in the second 6 months. One year follow-up confirm that alum-formulated recombinant human GAD65 is safe. Patients receiving 20 μg show no evidence of further decline in beta-cell function between 6 and 12 months. FIGS. 5A-5Y are graphs of results from this a phase II clinical study in accordance with one embodiment of the present invention.

In conclusion, the main outcomes of this phase II clinical trial support the clinical safety of the treatment and prevention methods of the present invention, together with evidence for the therapeutic efficacy of a 20 μg dose as reflected by an increase in both fasting and stimulated c-peptide. Evidence for this effect being mediated by an increase in regulatory T cells was also obtained.

Additional data was also obtained from an 18 month and 24 month follow-up study as described below.

Therapeutic Rationale

The majority of patients with insulin dependent (Type 1) diabetes, and also a 10% subset of non-insulin dependent (Type 2) diabetes patients (i.e. those with antibodies to GAD65), are currently recognised as having an autoimmune form of diabetes.

Although the contribution by the different components of the immune system is different for different autoimmune diseases, it is generally understood that the destructive process in autoimmune diabetes is orchestrated and executed by T lymphocytes, not antibodies. While this destructive process is primarily contributed to by autoreactive cytotoxic T lymphocytes (i.e. displaying the CD8+ cell surface marker) their activity is controlled by another lymphocyte subclass, T “helper” cells (instead displaying the CD4+ marker). T helper cells are therefore providing important regulatory functions in the activity of cytotoxic lymphocytes, and their manipulation provides a potent target for therapeutics to treat or prevent autoimmune diseases.

Interest in GAD65 in Type 1 diabetes stems from observations in the 1980s of the frequent occurrence (˜90%) of antibodies to GAD65 in patients with insulin-dependent diabetes. Since then, the clinical presence of GAD65 antibodies has become increasingly accepted as both a diagnostic and prognostic marker for this disease. Most importantly pre-clinical and clinical studies have confirmed the GAD65 protein as the most important autoantigen in the prediction and prevention of autoimmune diabetes.

In animal models for different autoimmune diseases, the appropriate administration of the autoantigen itself has been found capable of precipitating, as well as preventing, the associated autoimmune disease. These findings therefore provide strong support for the involvement of specific antigens in the aetiology of autoimmune diseases and also, conversely, of the possibility for “antigen-specific tolerisation therapies” in their treatment and cure.

Briefly, tolerisation involves the appropriate presentation of the autoantigen itself back to the immune system to enable an immune “re-programming” process to occur. It seems that the appropriate dose regimen, i.e. the quantity, route, frequency, adjuvant etc required for each autoantigen/autoimmune disease, is critical in determining which of several tolerisation mechanisms are activated. If the appropriate immune mechanism is engaged, then tolerisation to that autoantigen will occur and autoimmunity will be extinguished.

By way of background, in November 1993, two independent research groups in the U.S. simultaneously reported (in the scientific journal “Nature”) that administration of microgram quantities of GAD65 could induce tolerance and prevent insulin requirement in the NOD mouse pre-clinical model. Since then, the capability of GAD65 as a toleragen to prevent autoimmune diabetes has been confirmed experimentally in several independent laboratories. These include research groups at UCLA (Tian et al), Stanford (Tisch et al), Hopital Necker (Pleau et al) and University of Calgary (Yoon et al).

The immune mechanisms involved in GAD65 tolerisation in NOD mice have since been intensely investigated. Several published reports now support this type of immunomodulatory mechanism being evoked early in GAD65 tolerisation, and the down-regulation in autoimmunity that this induces is sufficient to preserve beta cell function and prevent exogenous insulin requirement.

Particularly because of close similarities in the NOD mouse model to its clinical counterpart, these pre-clinical findings support the possibility of rhGAD65 administration providing a clinical therapeutic for the prevention and treatment of autoimmune diabetes. Accordingly, administration of microgram quantities of alum-formulated rhGAD65 (referred to as “Alhydrogel-Diamyd™”) via an immunomodulatory “prime-and-boost” dose regimen to patients with autoimmune diabetes is proposed for therapeutic evaluation. The intended preservation of beta cell function is proposed to be clinically manifested by an increase in levels of insulin (or its surrogate: C-peptide) and result in prevention or delay in time to insulin requirement in diabetes patients with GAD65 antibodies.

Target Product Profile

The target product profile is supported by experimental data, but remains to be confirmed through continued clinical trials.

| Property | Target Product Profile |
|---|---|
| Indication | Indicated for the treatment of Type 1 diabetes |
| | patients and Type 2 diabetes patients |
| | with GAD antibodies |
| Contraindications | None identified in clinical trials conducted to date |
| Dosage | 20 μg |
| Dosage Regimen | Initial prime followed by a boost after one month. |
| | Additional boost after six months if indicated by a |
| | decrease in or unchanged C-peptide levels |
| | Yearly boosts thereafter if indicated by a decrease |
| | in C-peptide levels compared to previous visit. |
| | If decrease or unchanged C-peptide levels after |
| | three injections (one prime and two boosts) |
| | the treatment is terminated |
| Efficacy | >30% increase in C-peptide levels in fasting |
| | patients. Maintained or decreased existing insulin |
| | requirement (as a measure of beta cell function) |
| Shelf Life | 3 years |

Clinical Development

Clinical trials have been conducted with the Diamyd™ product available at different stages in development. The first clinical study used “laboratory grade” Diamyd™ Bulk Drug in a skin “prick test” study in selected volunteers. This was followed by a Phase I clinical trial in volunteers using GLP-grade Diamyd™ Bulk Drug from the commercial manufacturing process. The Phase II clinical trial in LADA patients has recently been completed with the (GMP) Alhydrogel-Diamyd™ formulation.

| | Study | | Individuals | | |
|---|---|---|---|---|---|
| Study | Location & | Study | (& | Study | Study |
| Type | CRO | dates | Number) | Endpoint(s) | Outcome |
| Skin | Sweden | 11 Feb. | Type 1 | Delayed | No DTH |
| Prick-Test | (Karolinska | 1995 | Diabetics | Type | Reactions to GAD |
| | Hospital) | | (7) | Hyper- | No AEs |
| | | | & Healthy | sensitivity | |
| | | | controls | (DTH) | |
| | | | (8) | | |
| Phase I | UK | Jan-Dec | Healthy | Safety/ | Safe |
| | (Pharmaco,-LSR) | 1999 | volunteers | Tolerability | Well-tolerated |
| | | | (24) | | |
| Phase II | Sweden | May 2001-April 2003 | LADA | Safety/ | No treatment |
| | (Chiltern | | Patients | Efficacy | related AEs |
| | International) | | (47) | | Efficacy in 20 μg |
| | | | | | dose group |
| | | | | | (C-peptide) |
| | | | | | Immuno- |
| | | | | | modulation |
| | | | | | demonstrated |

Phase II
Study Design

A Phase II randomized, double blind, placebo controlled, group comparison dose escalation study was performed in a total of 47 patients. The study was designed to assess both safety and efficacy of treatment with Alhydrogel-formulated Bulk drug (Diamyd™).

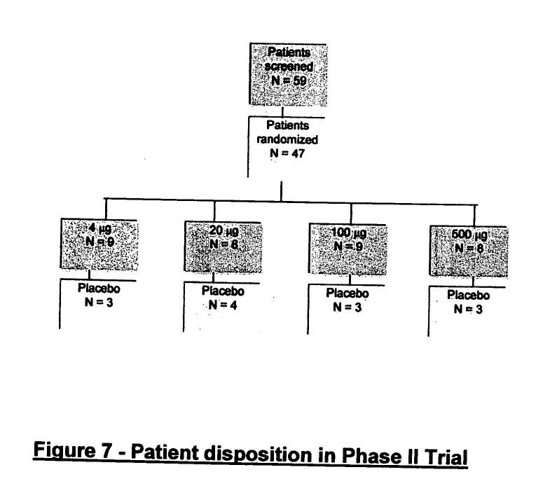

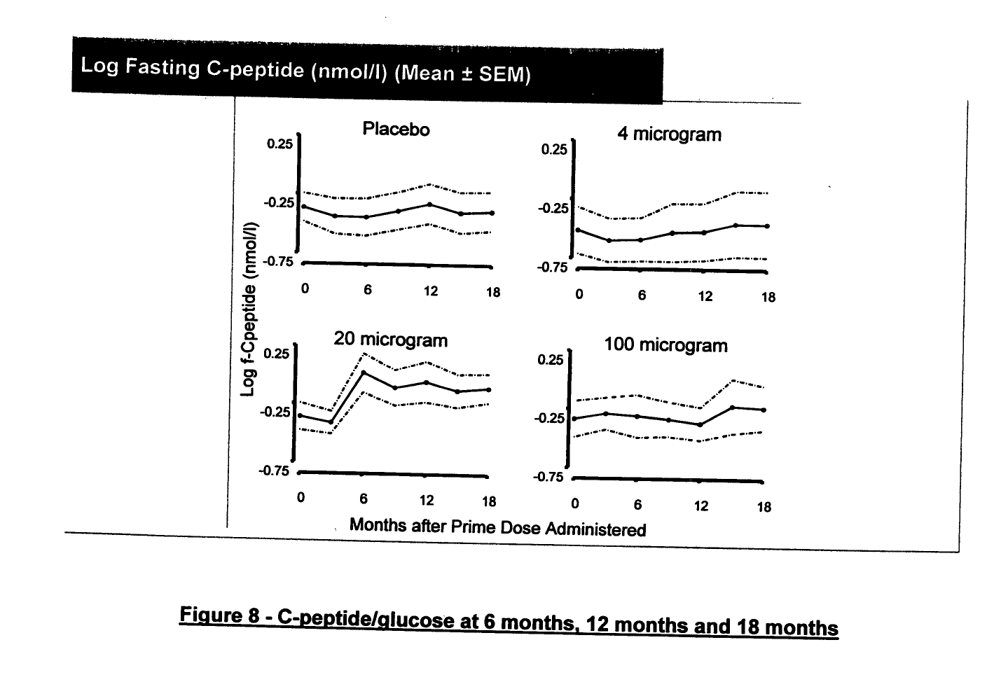

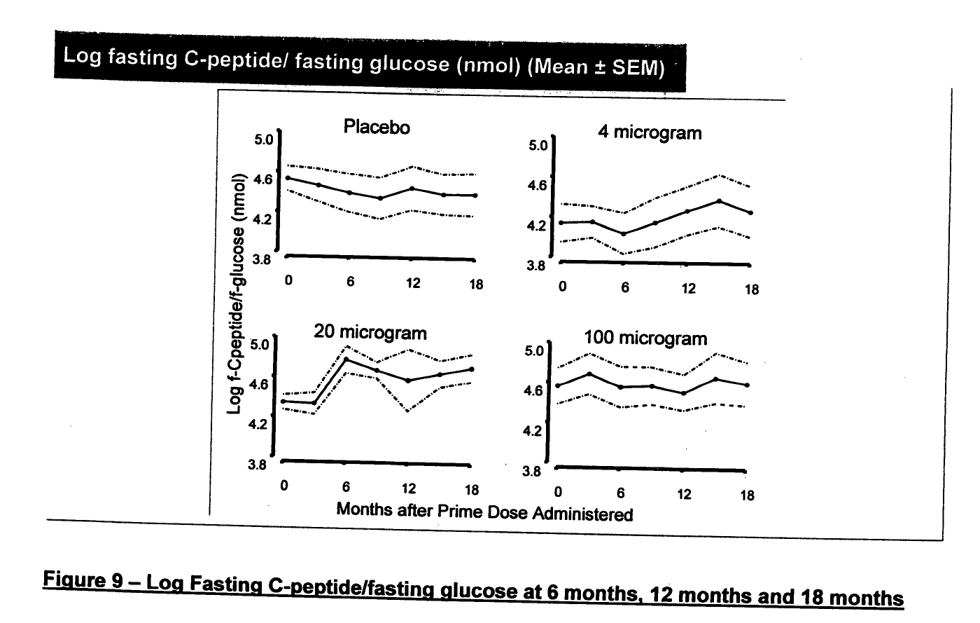

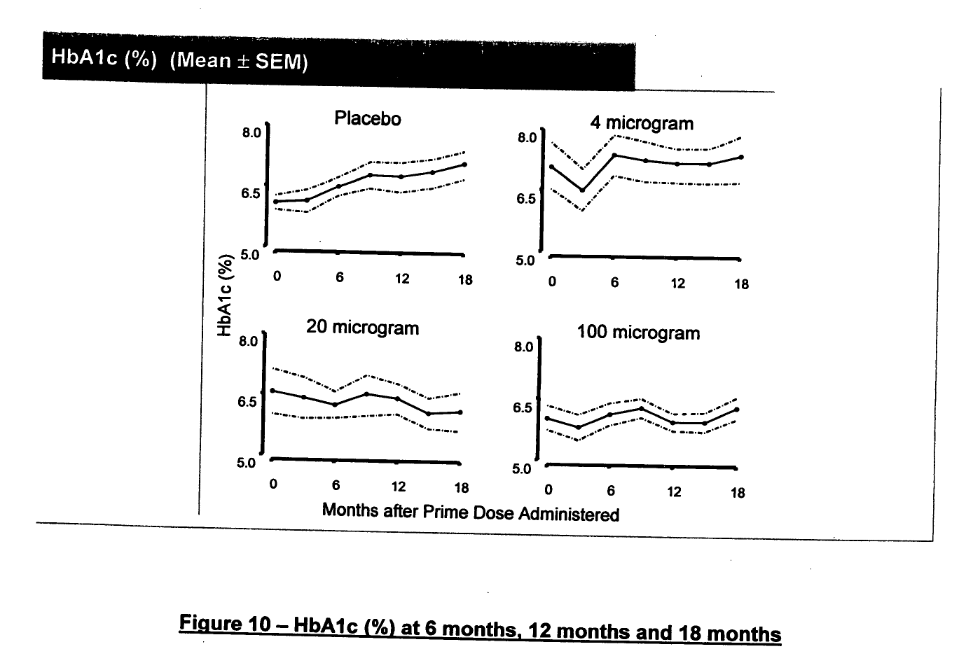

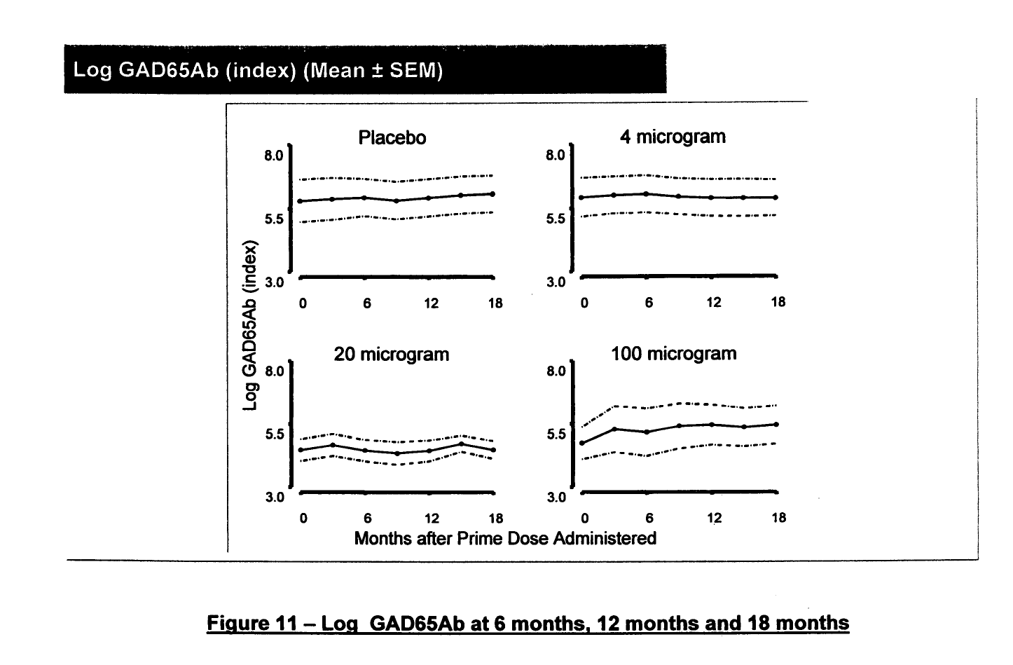

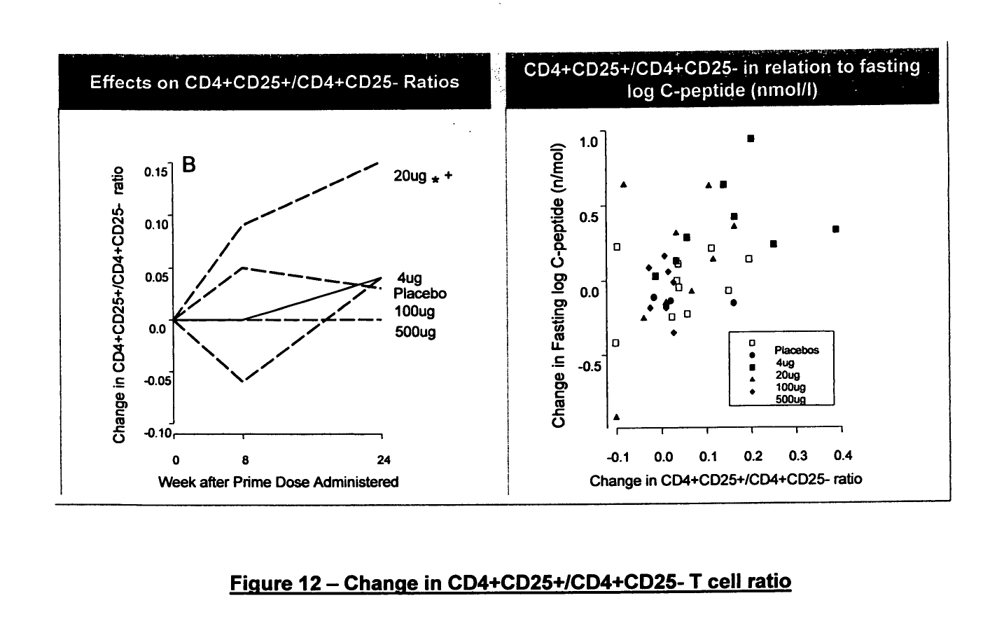

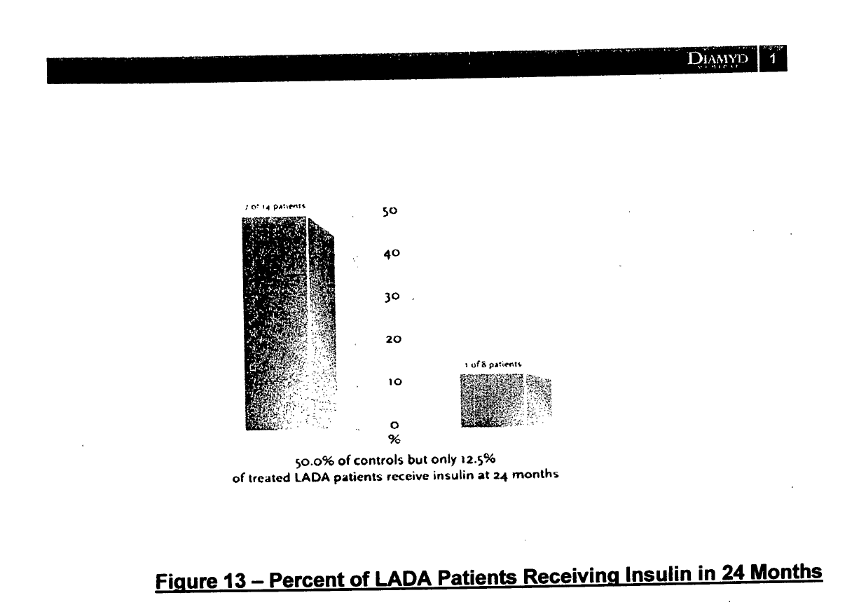

Patient disposition is shown in FIG. 7. There were 39 males (83.0%) and 8 female (17.0%) patients randomised in the trial. There was a similar number of female patients in the placebo, 4 μg, 20 μg, and 100 μg dose groups, however there were no female patients in the 500 μg dose group.

All study groups were comparable with regard to age, BMI, and basal levels of fasting glucose, fasting and meal-stimulated C-peptide, and HbA_{1c} as seen in Table 1 below.

Baseline Characteristics

| | | Group 1 | Group 2 | Group 3 | Group 4 |
|---|---|---|---|---|---|
| | Placebo | (4 μg) | (20 μg) | (100 μg) | (500 μg) |
| n | 13 | 9 | 8 | 9 | 8 |
| Age (years) | 56 (37-66) | 58 (39-69) | 57 (48-67) | 57 (30-69) | 53 (39-62) |
| Males (n) | 12 | 7 | 6 | 6 | 28 |
| Log GADA (U/ml) | 4.6 (3.3-13.3) | 5.0 (3.9-9.4) | 4.1 (3.5-7.3) | 3.8 (3.4-7.8) | 4.7 (3.3-7.5) |
| BMI (kg/m²) | 26 (23-32) | 27 (20-35) | 28 (23-33) | 27 (20-39) | 26 (21-33) |
| HbA1c (%) | 5.9 (4.7-7.4) | 6.7 (5.5-10.9) | 5.9 (5.1-9.9) | 6.0 (4.6-7.1) | 6.0 (5.4-8.1) |
| P-glucose (mmol/L) | | | | | |
| Pre-Sustacal | 7.8 (5.5-9.5) | 9.6 (5.9-15.8) | 9.1 (6.3-17.4) | 7.7 (6.2-9.0) | 9.1 (6.3-15.1) |
| P c-peptide (nmol/L) | | | | | |
| Pre-Sustacal ® | 0.67 (0.3-1.7) | 0.6 (0.3-1.6) | 0.7 (0.5-1.4) | 0.7 (0.3-1.5) | 0.6 (0.3-1.8) |
| Post-Sustacal ® | 1.6 (0.5-3.7) | 1.3 (0.7-2.9) | 1.5 (1.0-2.0) | 2.0 (0.6-3.9) | 1.3 (0.8-5.1) |
| IA-2A positive (n) | 1 | 0 | 2 | 1 | 1 |
| Total T-cells (×/10⁹/L) | 1.3 (0.7-2.0) | 1.1 (0.8-1.6) | 1.3 (0.8-2.0) | 2.0 (0.9-2.4) | 1.1 (0.6-2.0) |
| CD4+/CD8+ ratio | 1.7 (0.8-3.8) | 1.6 (0.7-9.3) | 1.2 (0.8-3.2) | 1.8 (1.1-4.9) | 1.9 (1.0-3.0) |
| CD4+ CD25+/ | 0.18 (0.05-0.27) | 0.20 (0.17-0.32) | 0.15 (0.08-0.27) | 0.20 (0.07-0.28) | 0.09 (0.03-0.35) |
| CD4+ CD25− ratio | | | | | |

Median values (range) are shown

Study Endpoints

Apart from routine safety variables such as Clinical Examination and Adverse Event reporting and assessment, special emphasis was put on evaluating the impact of treatment on the patients diabetic status.

Efficacy parameters included Blood Glucose, HbA_{1c} and C-peptide levels (both fasting and meal-induced). These were measured on day 1, and at 1, 2, 3 and 6 months after initial treatment. In addition, antibody and lymphocyte parameters were assessed to investigate the impact of treatment on the patients immune system. These included antibody assays for autoantigens recognised as being involved in the autoimmune diabetes (GAD65, insulin, IA-2, and ICA), and lymphocytes potentially involved in the autoimmune process. These assays included FACS on whole blood for lymphocyte subsets, ELISPOT analysis of frozen polymorphonuclear cells from blood, and ELISA for serum cytokines.

Immunoassays were performed on patient samples obtained from visits on day 1, and weeks 1, 4 (i.e. immediately pre-boost), 5 (i.e. 1 week post-boost), 8, and 24. These immunoassays will be performed at month 9 and 12 during the first 6 months of the 4.5 year study follow-up, and then for GAD Ab titre alone every 6 months for the remaining 4 years.

Standard Haematology and Biochemistry analyses were performed at screening, day 1, and weeks 1, 2, 4, 8, 12 and 24.

Clinical Safety

There were no SAEs or deaths during the trial. There were 32/47 patients (68%) with 51 AEs. The majority of patients in each treatment group had at least one AE, most of which were due to influenza-like symptoms, with nasopharyngitis as the most common AE.

Clinical Efficacy

C-Peptide at 6 Months, 12 Months and 18 Months:

A 36% (p=0.008) increase in fasting and a 19% (p=0.0156) increase in meal-stimulated C-peptide levels at week 24 were seen in the 20 μg dose group. The increase in fasting C-peptide was significantly different compared to placebo (p=0.0011). The effect appeared to be maintained troughout the study period (18 months) No other statistically significant changes in C-peptide were seen in the other dose groups.

The increase in fasting plasma C-peptide seen in the 20 μg dose group was also evident when the data were normalised for glucose and compared to either baseline (47% increase, p=0.0078) or placebo (p=0.0008). An increase in meal-stimulated C-peptide/glucose (24% increase, p=0.0391) compared to baseline was also observed within the 20 μg group.

Patients given placebo or the 4 μg dose had almost identical plasma C-peptide/glucose ratio during the 24 weeks follow-up, inferring the absence of impact in the 4 μg dose group and supporting this as being defined a “no-effect” dose level. However, over the 18 months follow-up period a small gradual increase in the C-peptide/glucose ratio was seen. This was however not statistically significant.

HbA_{1c}:

A steady gradual increase in HbA_{1c} levels (p=0.01) was found in the placebo but not in the other dose-groups, inferring a reduction in glycemic control as diabetes progressed in untreated patients through the 18 months study period. A greater trend towards elevated HbA_{1c} levels was indicated for the placebo, 4 μg dose group, than for the 100 μg dose group, suggesting, in contrast, an improvement in glycemic control in patients receiving the latter dose level. In the 20 μg dose group in particular a decrease in HbA_{1c} levels was seen throughout the 18 month follow-up period indicating an improvement in glycemic control.

The average rate of change in HbA_{1c} per month was statistically significant compared to placebo in the 20 μg dose group (P20₀₁) which was estimated by a linear mixed model assuming random intercepts and slopes.

Rate of change per month compared to placebo group

| | | 95% Cl | |
|---|---|---|---|
| Placebo group | Mean Difference | Reference | p-value |
| 4 μg dose group | −0.05 | (−0.12, 0.03) | 0.20 |
| 20 μg dose group | −0.09 | (−0.17, −0.02) | <0.01 |
| 100 μg dose group | −0.07 | (−0.14, 0.00) | 0.06 |

Impact on Patient Immune System:
GAD65 Antibodies:

No significant change in GAD65Ab levels (expressed as U/ml) was found in the placebo or 4 μg group. Levels in the 20 μg dose group did not change during the first 4 weeks, however, between 8 and 24 weeks 7/8 (88%) patients showed a decline. Similarly, in the 100 μg dose group there was a decline in 8/9 (89%) patients despite an increase in three of these patients between 4 and 24 weeks. All eight patients in the 500 μg group developed either an early increase before week 4 or a gradual increase after additional boost injections during the 24 weeks, and showed between a 2.1-fold and 22.1-fold maximum increase in GAD65Ab levels.

Measurement of GAD65Ab is currently the most robust marker used to identify autoimmune diabetes and predict future insulin requirement in LADA. Their clear induction in the top dose group (p=0.001) support the impact of Diamyd™ treatment on the immune system. The decrease in GAD65Ab levels associated with efficacy at the 20 μg dose level may reflect a preservation of beta cells. This may be rationalized by reduced beta cell destruction lowering the amount of GAD65 released and presented to the immune system, resulting in reduced quantities of antibodies produced. A direct down-modulatory effect on B lymphocyte activity is also possible.

T Lymphocytes:

There were no differences between the groups in the majority of peripheral T lymphocyte subsets (CD3+, CD4+, CD8+, NK) analysed on day 1 and during the 24 weeks of observation. In contrast however, a statistically significant increase over time was found for the ratio of a particular T cell subset in patients receiving 20 μg Diamyd™ (p=0.012), and this increase was different from that in the placebo group (p=0.03). This T lymphocyte subset (with the surface markers CD4+ and CD25+) are currently implicated in regulating the activity of other T cells, and their involvement in inhibition of autoimmunity has also been demonstrated in animal models.

A positive correlation between the change in this CD4⁺CD25⁺/CD4⁺CD25⁻ T cell ratio and change in fasting C-peptide (r=0.51; p<0.005) was found, supporting the increase in C-peptide being related to an increase in this T cell ratio. This correlation was also found for meal-stimulated C-peptide (r=0.34; p<0.05).

In view of the critical involvement of GAD65-specific T helper and cytotoxic T cells in the autoimmune destructive process leading to insulin dependence, positive evidence for the induction of a different T cell subset, capable of down-regulating autoimmunity, is considered to be an important study finding.

### CONCLUSIONS

The foregoing study supports the following conclusions:

Statistically significant increases in fasting and meal-stimulated C-peptide levels were apparent in the 20 μg dose, and these positive effects were confirmed when these data were normalised against fasting blood glucose. The positive effects was maintained throughout an 18 months follow up period.

Positive evidence for immunomodulation was provided by analysis of T cell subsets. This strongly indicates that two injections of Diamyd™ effectively increases C-peptide production as a result of specific down regulation of beta cell inflammation by activated regulatory T cells.

The Diamyd™ treatment raises no safety concerns. In particular, GAD65Ab levels, even after re-boosts at the top dose (4×500 μg), do not support the likelihood of Diamyd™ treatment causing neurological disease (e.g. Stiff Man).

At two years, 7/14 patients in a control group (a “no-effect” 4 μg dose group+placebo) versus 1/8 of patients receiving the effective 20 μg dose became insulin dependent.

Positive evidence for safety and efficacy together with the provision of insights into the mechanism of action of the treatment therefore strongly support the potential for Diamyd™ as a therapy for autoimmune diabetes.

All publications and patents mentioned herein are hereby incorporated by reference to the same extent as if each individual publication or patent was specifically and individually indicated to be incorporated by reference. Related Provisional Patent Application Ser. No. 60/478,392 and the corresponding U.S. patent application based thereupon is also hereby incorporated herein by reference. Many variations of the present invention within the scope of the appended claims will be apparent to those skilled in the art once the principles described herein are understood.

### REFERENCES

1. Gepts W. Pathologic anatomy of the pancreas in juvenile diabetes mellitus. Diabetes 1965; 14:619-633.
2. Leslie R D, Atkinson M A, Notkins A L. Autoantigens IA-2 and GAD in Type I (insulin-dependent) diabetes. Diabetologia 1999; 42:3-14.
3. Verge C F, Gianani R, Kawasaki E, et al. Number of autoantibodies (against insulin, GAD or ICA512/IA2) rather than particular autoantibody specificities determine risk of type I diabetes. J Autoimmun 1996; 9:379-383.
4. Graham J, Hagopian W A, Kockum I, et al. Genetic effects on age-dependent onset and islet cell autoantibody markers in type 1 diabetes. Diabetes 2002; 51:1346-1355.
5. Zimmet P Z, Tuomi T, Mackay I R, et al. Latent autoimmune diabetes mellitus in adults (LADA): The role of antibodies to glutamic acid decarboxylase in diagnosis and prediction of insulin dependency. Diabetic Med 1994; 11:299-303.
6. Turner R, Stratton I, Horton V, et al. UKPDS 25: autoantibodies to islet-cell cytoplasm and glutamic acid decarboxylase for prediction of insulin requirement in type 2 diabetes. UK Prospective Diabetes Study (UKPDS). Lancet 1997; 350:1288-1293.
7. Tuomi T, Carlsson, A Li H, et al. Clinical and genetic characteristics of type 2 diabetes with and without GAD antibodies. Diabetes 1999; 48:150-157.
8. Kaufman D L, Clare-Salzler M, Tian J, et al. Spontaneous loss of T-cell tolerance to glutamic acid decarboxylase in murine insulin-dependent diabetes. Nature 1993; 366:69-72.
9. Tisch R, Yang X D, Singer S M, Liblau R S, Fugger L, McDevitt H O. Immune response to glutamic acid decarboxylase correlates with insulitis in non-obese diabetic mice. Nature 1993; 366:72-75.
10. Pleau J M, Fernandez-Saravia F, Esling A, Homo-Delarche F, Dardenne M. Prevention of autoimmune diabetes in nonobese diabetic female mice by treatment with recombinant glutamic acid decarboxylase (GAD65). Clin Immunol Immunopathol. 1995; 76: 90-95.
11. Petersen J S, Karlsen A E, Markholst H, Worsaae A, Dyrberg T, Michelsen B. Neonatal tolerization with glutamic acid decarboxylase but not with bovine serum albumin delays the onset of diabetes in NOD mice. Diabetes 1994; 43:1478-1484.
12. Tian J Atkinson M A, Clare-Salzler M, Herschenfeld A, Forsthuber T, Lehmann P V, Kaufman D L. Nasal administration of glutamate decarboxylase (GAD65) peptides induces Th2 responses and prevents murine insulin-dependent diabetes. J Exp Med 1996a; 183:1-7.
13. Tian J, Clare-Salzler M, Herschenfeld A, et al. Modulating autoimmune responses to GAD inhibits disease progression and prolongs islet graft survival in diabetes-prone mice. Nat Med. 1996b; 2:1348-1353.
14. Tisch R, Liblau R S, Yang X D, Liblau P, McDevitt H O. Induction of GAD65-specific regulatory T-cells inhibits ongoing autoimmune diabetes in nonobese diabetic mice. Diabetes 1998; 47:894-899.
15. Lethagen Å L, Ericsson U B, Hallengren B, Groop L, Tuomi T. Glutamic acid decarboxylase antibody positivity is associated with an impaired insulin response to glucose and arginine in nondiabetic patients with autoimmune thyroiditis. J Clin Endocrinol Metab 2002; 87:1177-1183.
16. Bingley P J, Bonifacio E, Mueller P W. Diabetes antibody standardization program: first assay proficiency evaluation. Diabetes 2003; 52:1128-1136.
17. Brown P, Rothwell J C, Marsden C D. The stiff leg syndrome. J Neurol Neurosurg Psychiatry. 1997; 62: 31-37.
18. Barker R A, Revesz T, Thom M, Marsden C D, Brown P. Review of 23 patients affected by the stiff man syndrome: clinical subdivision into stiff trunk (man) syndrome, stiff limb syndrome, and progressive encephalomyelitis with rigidity. J Neurol Neurosurg Psychiatry 1998; 65: 663-640.
19. Smith G E, Summers M D, Fraser M J. Production of human beta interferon in insect cells infected with baculovirus expression vector. Mol Cell Biol. 1983; 3:2156-2165.
20. Shevach E M. CD4+ CD25+ suppressor T cells: more questions than answers. Nat Rev Immunol 2002; 2:389-400.
21. Sakaguchi S. Regulatory T cells: key controllers of immunologic self-tolerance. Cell 2000; 101:455-458.
22. Shevach E M, McHugh R S, Piccirillo CALIFORNIA, Thornton A M. Control of T-cell activation by CD4+ CD25+ suppressor T cells. Immunol Rev 2001; 182:58-67.
23. Jonuleit H, Schmitt E, Stassen M, Tuettenberg A, Knop J, Enk A H. Identification and functional characterization of human CD4(+)Cd24(+) T cells with regulatory properties isolated from peripheral blood. J Exp Med 2001; 193:1285-1294.
24. Stephens L A, Mottet C, Mason D, Powrie F. Human CD4(+)CD25(+) thymocytes and peripheral T cells have immune suppressive activity in vitro. Eur J Immunol 2001; 31:1247-1254.

The preferred embodiment herein disclosed is not intended to be exhaustive or to unnecessarily limit the scope of the invention. The preferred embodiments were chosen and described in order to explain the principles of the present invention so that others skilled in the art may practice the invention. Having shown and described preferred embodiments of the present invention, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

1. A method of treating diabetes in a human, said method comprising administering to a human an effective amount of a human recombinant GAD65 protein and at least one adjuvant for an effective time so as to stimulate the production of insulin in said human to a level above that existing prior to said administration.

2. A method according to claim 1 wherein said administration is subcutaneous.

3. A method according to claim 1 wherein said adjuvant is aluminum hydroxide.

4. A method according to claim 1 wherein said human recombinant GAD65 protein and said at least one adjuvant are administered in a dosage such that said human recombinant GAD65 protein is in the range of from about 10 to about 50 micrograms.

5. A method according to claim 1 wherein said human recombinant GAD65 protein and said at least one adjuvant are administered in a dosage such that said human recombinant GAD65 protein is in the range of from about 5 micrograms to about 500 micrograms.

6. A method according to claim 1 additionally comprising administering at least one booster dosage of said human recombinant GAD65 protein following the first administration of said human recombinant GAD65 protein, and wherein said booster is administered in a dosage such that said human recombinant GAD65 protein is in the range of from about 10 to about 50 micrograms.

7. A method according to claim 1 additionally comprising administering at least one booster dosage of said human recombinant GAD65 protein following the first administration of said human recombinant GAD65 protein, and wherein said booster is administered in a dosage such that said human recombinant GAD65 protein is in the range of from about 5 micrograms to about 500 micrograms.

8. A method for suppressing or reducing the immune response of a human to glutamic acid decarboxylase comprising administering to said human an effective immunosuppressive dose of human recombinant GAD65 protein in the range of from about 10 to about 50 micrograms administered with at least one adjuvant.

9. A method according to claim 8 wherein said administration is subcutaneous.

10. A method according to claim 8 wherein said adjuvant is aluminum hydroxide.

11. A method according to claim 8 additionally comprising administering at least one booster dosage of said human recombinant GAD65 protein following the first administration of said human recombinant GAD65 protein, and wherein said booster is administered in a dosage such that said human recombinant GAD65 protein is at a level in the range of from about 10 to about 50 micrograms.

12. A method according to claim 13 wherein the level of treatment response is determined through measurement of CD4⁺CD25⁺ lymphocytes prior to said at least one booster dosage.

13. A method to increase insulin production in a diabetes patient with beta cell antibodies, said method comprising administering to a human an effective amount of beta cell antigen in a pharmaceutical carrier for an effective time so as to stimulate the production of insulin in said human to a level above that existing prior to said administration.

14. A method according to claim 13 wherein said beta cell antigens include at least one component selected from the group consisting of: GAD65, GAD67, insulin, insulin-peptide, proinsulin, proinsulinpeptide, sulfatide, heat schock protein, S100 beta protein, IA-2, or any peptide, altered peptide ligand, chimeric molecule, or conjugated molecule or fragment thereof.

15. A method according to claim 13 wherein said administration is selected from the goup consisting of subcutaneous, intravenous, oral and gene therapy adminsitration.

16. A method to increase insulin production in a diabetes patient with beta cell antibodies, said method comprising administering to a human an effective amount of DNA or RNA nucleotides coding for at least one beta cell antigen, in a pharmaceutical carrier and for an effective time so as to stimulate the production of insulin in said human to a level above that existing prior to said administration.

17. A method according to claim 16 wherein said DNA or RNA nucleotides codes for at least one component selected from the group consisting of: GAD65, GAD67, insulin, insulin-peptide, proinsulin, proinsulinpeptide, sulfatide, heat schock protein, S100 beta protein, IA-2, or any peptide, altered peptide ligand, or by anti-sense oligos to at least one of said nucleotides, or any chimeric molecule, conjugate molecule or fragment thereof.

18. A method according to claim 17 wherein at least one said component is produced recombinantly in a prokaryotic expression system capable of posttranslational palmitoylation.

19. A method according to claim 18 wherein said expression system used to express said component is baculovirus grown in Spodotera frugiperda 9 (Sf9) cells.

20. A method according to claim 16 wherein said administration is selected from the goup consisting of subcutaneous, intravenous, oral and gene therapy administration.

21. A method according to claim 16, wherein said at least one component is administered in a dosage such that at least one of said components is in the range of from about 10 micrograms to about 50 micrograms.

22. A method according to claim 16, wherein said at least one component is administered in a dosage such that at least one of said components is in the range of from about 0.001 mgs/kg to about 0.1 mgs/kg.

23. A method according to claim 16 additionally comprising administering at least one booster dosage of said components following said administration, and wherein said booster is administered in a dosage such that at least one of said components is in the range of from about 10 micrograms to about 50 micrograms.

24. A method according to claim 16 additionally comprising administering intravenously at least one booster dosage of said components following said administration, and wherein said booster is administered in a dosage such that at least one of said components is in the range of from about 0.001 mgs/kg to about 0.1 mgs/kg.

25. A pharmaceutical composition for treatment of diabetes comprising of at least one of the components beta cell antigens include at least one component selected from the group consisting of: GAD65, GAD67, insulin, insulin-peptide, proinsulin, proinsulinpeptide, sulfatide, heat schock protein, S100 beta protein, IA-2, or any peptide, altered peptide ligand, chimeric molecule, or conjugated molecule or fragment thereof, said at least one component produced recombinantly in a prokaryotic expression system capable of posttranslational palmitoylation.

26. A pharmaceutical composition according to claim 25 additionally comprising alum and a Zwittergent present in a concentration relation to said at least one said component of from about 1:1 to about 1:8.

27. A pharmaceutical composition according to claim 25 additionally comprising human serum abumin and a Zwittergent present in a concentration relation to said at least one said component of from about 1:1 to about 1:8.

28. A method to increase beta cell mass in a diabetes patient with beta cell antibodies, said method comprising administering to a human an effective amount of beta cell antigen in a pharmaceutical carrier for an effective time so as to allow improved survival of regenerated beta cells as such beta cells are exposed to a lower autoimmune attack than they should have been without such administration.

29. A method according to claim 28 wherein said beta cell antigens include at least one component selected from the group consisting of: GAD65, GAD67, insulin, insulin-peptide, proinsulin, proinsulinpeptide, sulfatide, heat schock protein, S100 beta protein, IA-2, or any peptide, altered peptide ligand, chimeric molecule, or conjugated molecule or fragment thereof.

30. A method according to claim 28 wherein administration of said beta cell antigen is made in connection with administration of a substance capable of assisting beta cell regeneration.

31. A method according to claim 28 where said substance capable of assisting beta cell regeneration is at least one from the group: antiCD3-antibodies; antiCD25-antibodies; GLP-1 (Novo); NN2211 (Lilly); AC2993 (Amylin); AC2993LAR (Amylin), Betatropin (Restoragen); Glugagon-like peptide, PPAR-gamma agonist; Dual PPAR agonist; Galida (Astra-Zeneca) and Metformin.

32. A method according to claim 28 where said substance capable of assisting beta cell regeneration is administered during the period from 8 weeks prior to 8 weeks after administration of the beta cell antigen.
